SEQUENCE LISTING (10 sequences)

SEQ ID NO: 1 - DNA, 2448 bp, Drosophila melanogaster
atgacccacaatcttggtatggcgccatatcgattgccgggtccagcgggcggactctgtccgcccgattttaagccgccgcctcccacggacatcatctcggcgccgagcaatccgaagaagcggcgaaaaacctcaagtgccgccaactccgctgcagcggtggctgcggcggcggctgcagcagctgctgcgaattccatgcagcagcagcaggcgccacccacaccgcaggatttgctgccccctccgccaatgggaggcttcggagacaccattattgcctcgaatccattcgacgacagtccccaggtgtcggcgatgtccagctcagcggccgcggcgatggcggccatgaatcagatgggcggcggaccaggaggtggtcactttggcggcggtggaccgggtgggcacccgcactgggaagaccgcatgggcatgggcggtggacctcctcccccgcctcacatgcatccccatatgcacccgcatcatccaggcggacctatgggtcacccacatggcccacatccgcacatgggtggtccacctccaatgcgaggaatgagccccatgcacccccatcaaatgggaccgggaccaggcgtcggactaccgccgcatatgaatcacggaaggccagggggacctggtggtcctggaggacccgtcccaatgggtagtcccatgggtggaatagctggcatgggcggcatgagcccaatgggcggaatgggaggccccagcatatcaccccatcacatgggcatgggtggtctgtcgcccatgggaggcggtcccaacggacccaatccgcgagccatgcagggttcaccgatgggcggtccggggcagaactcgccaatgaactcactgcctatgggttcgccaatgggcaatccaattggcagcccgttgggccctccctcgggaccgggccctgggaatcccggcaataccggcggaccacagcagcaacaacaacaacctccgcagccaccgatgaacaacgggcagatgggtcctcctcctctgcacagtccgctcggaaacggaccaacgggtcatggcagtcacatgcctggaggaccaatcccaggaccaggtcctgggcctggcggcctagtaggtcccggtggcatctcccccgcgcacggcaataacccgggtggttctgggaacaacatgctcggcgggaatcccggcggcggcaacagcaacaacaacggaagcaatacaagtaacgccagcaacaacaatcaaaatcctcacctctcgccagcagccggacgcctgggagtgccgacgtcgatgcagtcgaatggaccttcggtatcatcggtagcctcctcatcggttccctcgcccgccacgcccacgctcacgcccacatcgacggccacgtccatgtccacgtcagtgcctacatcctcgccagcgccgcccgccatgtcaccgcatcactcgctaaacagcgccggcccgagtccgggcatgcccaactcgggacccagcccgctgcagtcaccagccggacccaatggccccaataacaacaacagcaataacaacaacggacccatgatgggccagatgatcccgaacgcagttcctatgcagcaccagcagcacatgggcggcggcccacctggccacgggcccggaccaatgcccggaatgggcatgaaccaaatgctgccaccgcagcaaccctcccatcttggtcccccgcatccgaatatgatgaaccacccgcatcatccgcaccatcatcctggcggaccaccgccgcacatgatgggtggacccggaatgcacggcggtcctgctggaatgcctcctcatatgggcggaggacctaatccgcacatgatgggcggtccgcacgggaacgcgggtccgcacatgggccacggccacatgggtggagtaccaggtccaggacccggacccggcggcatgaacggacccccgcatccgcacatgtccccgcaccacggacatccgcatcaccaccacaatccgatgggcggcccaggtccaaatatgttcggcggtggtggaggaggtcccatgggtcccggtggaccgatgggcaacatggggcccatgggaggtggcccgatgggcggccctatgggcgtaggtcccaagccgatgacaatgggcggcgggaagatgtacccgccgggacagccaatggtctttaatccgcagaacccgaatgcgccgcccatatatccttgtggcatgtgccacaaggaggtgaacgacaacgacgaagccgtgttctgtgaatccggttgtaactttttctttcacagaacctgtgttggcctgacagaggcggccttccaaatgctcaacaaggaggtgtttgccgagtggtgctgcgacaagtgcgtgtcttccaagcatattcccatggtcaagttcaagtgttga

SEQ ID NO: 2 - DNA, 1366 bp, Homo sapiens
ggatccccacatgcccgccgagaactctccagctcccgcttacaaagtttcctcgcatggtggtgatagtggactggatgggttaggaggaccaggtgtacaactaggaagcccagataagaaaaagcgcaaggcaaatacacagggaccttctttccctccattgtctgagtatgctccaccaccgaatccaaactctgaccatctagtggctgctaatccatttgatgacaactataatactatttcctataaaccactaccttcgtcaaatccatatcttggccctggttatcctggctttggaggctatagtacattcagaatgccacctcacgttcccccaagaatgtcttccccatactgtggtccttactcactcaggaaccagccacacccatttcctcagaatcctctgggcatgggttttaatcgacctcatgcttttaactttgggccacatgataattcaagtttcggtaatccatcttataataatgcactaagtcagaatgtcaacatgcctaatcaacattttagacaaaatcctgctgaaaatttcagtcaaattcctccacagaatgctagccaagtttctaaccccgatttggcatctaattttgttcctggaaataattcaaattttacttctccgttagaatctaatcattcttttattcctcccccaaacacttttggtcaagcaaaagcaccacccccaaaacaagactttactcaaggagcaaccaaaaacactaatcaaaattcctctgctcatccacctcacttgaatatggatgacacagtgaatcagagtaatattgaattaaaaaatgttaatcgaaacaatgcagtaaatcaggagaacagccgttcaagtagcactgaagccacaaacaataaccctgcaaatgggacgcagaataagccacgacaaccaagaggtgcagcagatgcctgcaccacagaaaaaagcaataaatcctctcttcacccaaaccgtcatggccattcgtcttctgacccagtgtatccttgtggaatttgtacaaacgaggtgaacgatgatcaggatgccatcttatgtgaggcctcttgtcagaaatggtttcatcggatctgtactggaatgactgaaacagcttatggcctcttaactgcagaagcatctgcagtatggggctgtgatacctgtatggctgacaaagatgtccagttaatgcgtactagagaaacttttggtccatctgcagtgggcagtgatgcttaatcaaaggcattaactaaagtgggtttattttcctgtgcattgcagaagttcattgacacaggattttaatgttttacattatttttttaaatgcat

SEQ ID NO: 3 - DNA, 1449 bp, Homo sapiens
cccgggtcccccactccatggccgcctcggcgccgcccccaccggacaagctggagggaggtggcggccccgcaccgccccctgcgccgcccagcaccgggaggaagcagggcaaggccggtctgcaaatgaagagtccagaaaagaagcgaaggaagtcaaatactcagggccctgcatactcacatctgacggagtttgcaccacccccaactcccatggtggatcacctggttgcatccaacccttttgaagatgacttcggagcccccaaagtgggggttgcagcccctccattccttggcagtcctgtgcccttcggaggcttccgtgtgcaggggggcatggcgggccaggtacccccaggctacagcactggaggtggagggggcccccagccactccgtcgacagccaccccccttccctcccaatcctatgggccctgctttcaacatgcccccccagggtcctggctacccacccccaggcaacatgaactttcccagccaacccttcaaccagcctctgggtcaaaactttagtcctcccagtgggcagatgatgccgggcccagtggggggatttggtcccatgatctcacccaccatgggacagcctcccagagcagagctgggcccaccttctctgtcccaacgatttgctcagccaggggctccttttggcccttctcctctccagagacctggtcaggggctccccagcctgccgcctaacacaagtccctttcctggtccggaccctggctttcctggccctggtggtgaggatggggggaagcccttgaatccacctgcttctactgcttttccccaggagccccactcaggctccccggctgctgctgttaatgggaaccagcccagtttccccccgaacagcagtgggcggggtgggggcactccagatgccaacagcttggcaccccctggcaaggcaggtgggggctccgggccccagcctcccccaggcttggtgtacccatgtggtgcctgtcggagtgaggtgaacgatgaccaggatgccattctgtgtgaggcctcctgccagaaatggttccaccgtgagtgcacaggcatgactgagagcgcctatgggctgctgaccactgaagcttctgccgtctgggcctgcgatctctgcctcaagaccaaggagatccagtctgtctacatccgtgagggcatggggcagctggtggctgctaacgatgggtgacgctggtgaagtggcccagggaagtgcacatgtctctccctgctcttccagggtgatttttttgatgtttggctcttggtccttgtttccactggctttccatccccatggggcagaaacagtggctcctgggagcagaaaaggaattgaggtgggcaggcagaagagcctggattgctcactgttttgggaaacttacatgttgagatct

SEQ ID NO: 4 - DNA, 1254 bp, Mus musclus
atgtcagcggaacaggacaaggagcccatcgcgctgaagagagttagaggtggtgacagtggactggatgggttaggagggcccaatatacaactcggaagcccagataagaaaaaacgcaaggccaacacacagggatcttcctttcctccgctgtcagagtacgcgccaccaccgaatccaaactccgaccatctagtggctgccaatccgttcgatgacagctacaacaccatttcctataaaccactgccttcatctaatccatatcttggccctgggtatcctggctttggaggctacagcacattcagaatgccaccccacgtccctccaagaatgtcttctccctactgtggtccttactcactcaggaatcagccccacccatttcctcagaatccgttgggcatgggctttaaccggcctcatgcttttaactttgggccgcatgataattcgaattttggaaacccaccttataataatgtactgactcaggacattaacatgcccggtcagcattttagacaaggctctgctgaaaacttcagtcagattcccccgcagaatgttggccaagtgtctaaccctgacctcgcatctaattttgcccctgggaataattcaaattttacctctccgttagaaacgaatcattcgtttattccacccccaaacgcgtttggccaagcaaaagctccacttcccaaacaagacttcactcaaggggcaaccaaaaccccgaatcagaattcgtccactcacccacctcacctaaatatggaggatccagtcaatcagagtaacgtcgagttaaaaaatgtcaacagaaacaacgttgtccaagagaacagccgttcgggcagcgcagaggccaccaacaaccatgcgaatgggacccagaacaagccccggcagcccaggggcgcagctgacctgtgcacccccgacaaaagccgcaagttctccctgctccccagccggcatggccattcctcctctgaccctgtgtacccgtgcgggatttgtacaaatgaagtgaatgacgatcaggacgccattctgtgtgaagcctcttgtcagaagtggtttcatcgcatctgcactggaatgaccgaaacagcctacgggctcctgacagcggaagcatccgcagtgtggggctgtgacacgtgcatggctgacaaggatgtccagctcatgcgcactagagaggcctttggtccacctgccgtgggcggcgatgcctaa

SEQ ID NO: 5 - DNA, 1221 bp, Mus musclus
atggccgcctcggcgccgcccccaccggacaagctggagggaggcagcggccccgcaccgccccccgcgccgcccagcaccgggaggaagcagggcaaggccggtctgcaaatgaagagccctgaaaagaagcgaagaaagtccaatactcagggtcctgcatattcacatctgacggagttcgccccacccccgacccccatggtggatcatctggtcgcttctaacccttttgaggatgatttcggagcccctaaagtggggggcgcaggccctccgttcctcggcagtccggtgccctttggaggctttcgtgtacaggggggcatggcaggccaggtacccccaagctacggcactggaggaggagggggtccccagccacttcgtcggcagccccctccttttccccccagccctatgggtccagcttttaatatgccccctcagggtccctggggtaccccgccccctggcaacatgaactttcccagtcaacccttcaaccagtctctgggccaaaactttagcccacctggtgggcaggtgatgccaggcccagtaggcggatttggtcccatgatctcaccgaccatgggacagcctcctagaggggagctgggtcctcctcctctcccccaacgctttacccaaccaggagcaccttatggtccttctcttcagagacctggtcagggactcacccagctgccctccaacacaagtcccttccctggtccagaccctggttttcctggacctggcggtgaggatggtgggaagcccttgaacccaccggctcccaccgcctttccccaggaagcaccattcgggctccccgctgctgctgtcaatgggaatcagcccagtttcccccctagcagcagtggtcgaggtgggggcactccagatgccaacagtctggcaccccccggcaaggcagggggaggctcagggccccagcctcccccaggcctggtgtacccctgcggtgcctgccgtagtgaggtaaatgatgaccaggatgccattctgtgtgaggcctcctgccagaagtggtttcaccgcgagtgcaccggcatgaccgagagtgcctacggcctgctgaccaccgaggcctctgccgtctgggcctgtgatctttgcctcaagaccaaggagatccagtctgtctacatccgagagggcatgggccagttggtggctgctaacgatgggtga

SEQ ID NO: 6 - protein, 815 aa, Drosophila melanogaster
MTHNLGMAPYRLPGPAGGLCPPDFKPPPPTDIISAPSNPKKRRKTSSAANSAAAVAAAAAAAAAANSMQQQQAPPTPQDLLPPPPMGGFGDTIIASNPFDDSPQVSAMSSSAAAAMAAMNQMGGGPGGGHFGGGGPGGHPHWEDRMGMGGGPPPPPHMHPHMHPHHPGGPMGHPHGPHPHMGGPPPMRGMSPMHPHQMGPGPGVGLPPHMNHGRPGGPGGPGGPVPMGSPMGGIAGMGGMSPMGGMGGPSISPHHMGMGGLSPMGGGPNGPNPRAMQGSPMGGPGQNSPMNSLPMGSPMGNPIGSPLGPPSGPGPGNPGNTGGPQQQQQQPPQPPMNNGQMGPPPLHSPLGNGPTGHGSHMPGGPIPGPGPGPGGLVGPGGISPAHGNNPGGSGNNMLGGNPGGGNSNNNGSNTSNASNNNQNPHLSPAAGRLGVPTSMQSNGPSVSSVASSSVPSPATPTLTPTSTATSMSTSVPTSSPAPPAMSPHHSLNSAGPSPGMPNSGPSPLQSPAGPNGPNNNNSNNNNGPMMGQMIPNAVPMQHQQHMGGGPPGHGPGPMPGMGMNQMLPPQQPSHLGPPHPNMMNHPHHPHHHPGGPPPHMMGGPGMHGGPAGMPPHMGGGPNPHMMGGPHGNAGPHMGHGHMGGVPGPGPGPGGMNGPPHPHMSPHHGHPHHHHNPMGGPGPNMFGGGGGGPMGPGGPMGNMGPMGGGPMGGPMGVGPKPMTMGGGKMYPPGQPMVFNPQNPNAPPIYPCGMCHKEVNDNDEAVFCESGCNFFFHRTCVGLTEAAFQMLNKEVFAEWCCDKCVSSKHIPMVKFKC

SEQ ID NO: 7 - protein, 419 aa, Homo sapiens
MPAENSPAPAYKVSSHGGDSGLDGLGGPGVQLGSPDKKKRKANTQGPSFPPLSEYAPPPNPNSDHLVAANPFDDNYNTISYKPLPSSNPYLGPGYPGFGGYSTFRMPPHVPPRMSSPYCGPYSLRNQPHPFPQNPLGMGFNRPHAFNFGPHDNSSFGNPSYNNALSQNVNMPNQHFRQNPAENFSQIPPQNASQVSNPDLASNFVPGNNSNFTSPLESNHSFIPPPNTFGQAKAPPPKQDFTQGATKNTNQNSSAHPPHLNMDDTVNQSNIELKNVNRNNAVNQENSRSSSTEATNNNPANGTQNKPRQPRGAADACTTEKSNKSSLHPNRHGHSSSDPVYPCGICTNEVNDDQDAILCEASCQKWFHRICTGMTETAYGLLTAEASAVWGCDTCMADKDVQLMRTRETFGPSAVGSDA

SEQ ID NO: 8 - protein, 406 aa, Homo sapiens
MAASAPPPPDKLEGGGGPAPPPAPPSTGRKQGKAGLQMKSPEKKRRKSNTQGPAYSHLTEFAPPPTPMVDHLVASNPFEDDFGAPKVGVAAPPFLGSPVPFGGFRVQGGMAGQVPPGYSTGGGGGPQPLRRQPPPFPPNPMGPAFNMPPQGPGYPPPGNMNFPSQPFNQPLGQNFSPPSGQMMPGPVGGFGPMISPTMGQPPRAELGPPSLSQRFAQPGAPFGPSPLQRPGQGLPSLPPNTSPFPGPDPGFPGPGGEDGGKPLNPPASTAFPQEPHSGSPAAAVNGNQPSFPPNSSGRGGGTPDANSLAPPGKAGGGSGPQPPPGLVYPCGACRSEVNDDQDAILCEASCQKWFHRECTGMTESAYGLLTTEASAVWACDLCLKTKEIQSVYIREGMGQLVAANDG

SEQ ID NO: 9 - protein, 417 aa, Mus musclus
MSAEQDKEPIALKRVRGGDSGLDGLGGPNIQLGSPDKKKRKANTQGSSFPPLSEYAPPPNPNSDHLVAANPFDDSYNTISYKPLPSSNPYLGPGYPGFGGYSTFRMPPHVPPRMSSPYCGPYSLRNQPHPFPQNPLGMGFNRPHAFNFGPHDNSNFGNPPYNNVLTQDINMPGQHFRQGSAENFSQIPPQNVGQVSNPDLASNFAPGNNSNFTSPLETNHSFIPPPNAFGQAKAPLPKQDFTQGATKTPNQNSSTHPPHLNMEDPVNQSNVELKNVNRNNVVQENSRSGSAEATNNHANGTQNKPRQPRGAADLCTPDKSRKFSLLPSRHGHSSSDPVYPCGICTNEVNDDQDAILCEASCQKWFHRICTGMTETAYGLLTAEASAVWGCDTCMADKDVQLMRTREAFGPPAVGGDA

SEQ ID NO: 10 - protein, 406 aa, Mus musclus
MAASAPPPPDKLEGGSGPAPPPAPPSTGRKQGKAGLQMKSPEKKRRKSNTQGPAYSHLTEFAPPPTPMVDHLVASNPFEDDFGAPKVGGAGPPFLGSPVPFGGFRVQGGMAGQVPPSYGTGGGGGPQPLRRQPPPFPPSPMGPAFNMPPQGPWGTPPPGNMNFPSQPFNQSLGQNFSPPGGQVMPGPVGGFGPMISPTMGQPPRGELGPPPLPQRFTQPGAPYGPSLQRPGQGLTQLPSNTSPFPGPDPGFPGPGGEDGGKPLNPPAPTAFPQEAPFGLPAAAVNGNQPSFPPSSSGRGGGTPDANSLAPPGKAGGGSGPQPPPGLVYPCGACRSEVNDDQDAILCEASCQKWFHRECTGMTESAYGLLTTEASAVWACDLCLKTKEIQSVYIREGMGQLVAANDG

(19) United States
(12) Patent Application Publication
(10) Pub. No.: US 2004/0209264 A1
(43) Pub. Date: Oct. 21, 2004

(54) New essential downstream component of the wingless signalling pathway

(76) Inventors: Thomas Kramps, Zurich (CH); Konrad Basler, Kusnacht (CH)

Correspondence Address: BROWDY AND NEIMARK, P.L.L.C., 624 NINTH STREET, NW, SUITE 300, WASHINGTON, DC, 20001-5303, US

(21) Appl. No.: 10/472,385

(22) PCT Filed: Feb. 1, 2002
(86) PCT No.: PCT/CH02/00063

Related U.S. Application Data
(60) Provisional application No. 60/277,976, filed on Mar. 23, 2001.

(51) Int. Cl. C12Q 1/68; C07H 21/04; C12N 5/06; C07K 14/435

(52) U.S. Cl. 435/6; 435/69.1; 435/320.1; 435/348; 530/350; 536/23.5

(57) ABSTRACT

The present invention relates to a new essential downstream component of the wingless signaling pathway. In particular, the invention relates to nucleotide sequences of the Drosophila melanogaster daughter of legless (doll) gene, of its encoded proteins, as well as derivatives, fragments and analogues thereof. The invention includes vertebrate and invertebrate homologues of the Doll protein, comprising proteins that contain a stretch of amino acids with similarity to the Drosophila Doll gene. Methods for producing the Doll protein, derivatives and analogs, e.g. by recombinant means, and antibodies to Doll are provided by the present invention as well. The invention also relates to methods for performing high throughput screening assays for compounds modulating Doll function in the Wnt pathway.

The present invention relates to a new essential downstream component of the wingless signaling pathway. In particular, the invention relates to nucleotide sequences of the Drosophila melanogaster daughter of legless (doll) gene, of its encoded proteins, as well as derivatives, fragments and analogues thereof. The invention includes vertebrate and invertebrate homologues of the Doll protein, comprising proteins that contain a stretch of amino acids with similarity to the Drosophila Doll gene. Methods for producing the Doll protein, derivatives and analogs, e.g. by recombinant means, and antibodies to Doll are provided by the present invention as well. The invention also relates to methods for performing high throughput screening assays for compounds modulating Doll function in the Wnt pathway.

### BACKGROUND OF THE INVENTION

Wnt genes encode a large family of secreted, cystein rich proteins that play key roles as intercellular signaling molecules in a wide variety of biological processes (for an extensive review see (Wodarz and Nusse 1998). The first Wnt gene, mouse wnt-1, was discovered as a proto-oncogene activated by integration of mouse mammary tumor virus in mammary tumors (Nusse and Varmus 1982). Consequently, the involvement of the Wnt pathway in cancer has been largely studied. With the identification of the Drosophila polarity gene wingless (wg) as a wnt-1 homologue (Cabrera, Alonso et al. 1987; Perrimon and Mahowald 1987; Rijsewijk, Schuermann et al. 1987), it became clear that wnt genes are important developmental regulators. Thus, although at first glance dissimilar, biological processes like embryogenesis and carcinogenesis both rely on cell communication via identical signaling pathways. In a current model of the pathway, the secreted Wnt protein binds to Frizzle cell surface receptors and activates the cytoplasmic protein Dishevelled (Dsh). Dsh then transmits the signal to a complex of several proteins, including the protein kinase Shaggy/GSK3, the scaffold protein Axin and β-Catenin, the vertebrate homologue of Armadillo. In this complex β-Catenin is targeted for degradation after being phosphorylated by Sgg. After Wnt signaling and the resulting down-regulation of Sgg activity, β-Catenin (or its Drosophila homologue Armadillo) escape from degradation and accumulate into the cytoplasm. Free cytoplasmic β-Catenin translocates to the nucleus by a still obscure mechanism, and modulates gene transcription through binding the Tcf/Lef family of transcription factors (Grosschedl R 1999).

This set up, in which the key transducer is continuously held in check, is highly susceptible to mutations in its inhibitory components. The loss of any of the three elements of the β-Catenin destruction complex leads to an increase in β-Catenin levels, and hence to the constitutive activation of the pathway. While this may reduce cellular viability, as upon loss of GSK-3 function, it can also lead to cell fate changes, uncontrolled proliferation and tumorigenic behavior as in the cases of APC and Axin (Barker N 1999; Morin 1999; Potter 1999; Roose and Clevers 1999; Waltzer and Bienz 1999). Attempts to counter these harmful situations must aim at curbing the nuclear activities of β-Catenin, either by preventing the formation of the β-Catenin-TCF complex or by interfering with its transcriptional activator function.

Currently, there are no known therapeutic agents effectively inhibiting β-Catenin transcriptional activation. This is partly due to the fact that many of the essential components required for its full activation and nuclear translocation are still unknown. consequently, there is an urge to understand more about this pathway in order to be able to develop effective drugs against these highly malignant diseases.

In order to identify new components required for Wingless activation the inventors used a Drosophila genetic approach to screen for dominant suppressors of the rough eye phenotype caused by ectopic expression of Wingless, the Drosophila homologue of Wnt, during eye development. Three genes were identified: the β-catenin homologue armadillo (arm), the tcf/lef-1 homologue pangolin (pan) and legless (lgs), a completely new gene (U.S. Ser. No. 09/915,543). The lgs gene was subsequently cloned and its in vivo requirement for Wingless signal transduction in embryo and in developing tissues was confirmed. The presence of Lgs is required for a transcriptional active Arm/Pangolin complex and over-expression of Lgs strongly stimulates the transcriptional output of this bipartite transcription factor. The human genome contains at least two human Lgs homologues. One of them, Bcl9, has been previously implicated in B cell malignancies (Willis, Zalcberg et al. 1998). It was also genetically and biochemically demonstrated that digs and hLgs bind to Armadillo and β-Catenin and are functionally required for Wnt signal propagation in human cells. However, genetic experiments strongly suggested the presence of a second protein which binds to Lgs and is essential for the function of the active β-Catenin-Pangolin-Lgs complex.

The present invention describes the cloning and functional characterization of a novel Drosophila protein, named Daughter of Legless (Doll), which binds to Lgs and is required for Wnt signaling. In addition, the invention provides the sequences of the functional and structural human and mouse homologues as well as methods to screen for compounds inhibiting Doll function in the Wnt pathway.

### DEFINITIONS

The term “Doll polypeptide”, “Doll protein” when used herein encompasses native invertebrate and vertebrate Doll and Doll variant sequences (which are further defined herein).

A “wild type sequence Doll” comprises a polypeptide having the same amino acid sequence as a Doll protein derived from nature. Such wild type sequence of Doll can be isolated from nature or produced by recombinant and/or synthetic means. The term “wild type sequence Doll” specifically encompasses naturally occurring truncated forms, naturally occurring variant forms (e.g., alternatively spliced forms) and naturally occurring allelic variants of Doll. In one embodiment of the invention, the wild type Doll sequence is a mature or full-length Doll sequence comprising amino acids 1 to 815 of dDoll (FIG. 1), or 1 to 419 of hDoll-1, or 1 to 406 of hDoll-2 (FIG. 2), or 1 to 417 of mDoll-1, or 1 to 407 of mDoll-2 (FIGS. 3). “Doll variant” means an active Doll, having at least about 50% amino acid sequence identity with the amino acid sequence of a wild type Doll protein of FIG. 1, 2 and 3. The term “Doll variant” however, does also include functional homologues of Doll in the Wnt pathway.

“Percent (%) amino acid sequence identity” with respect to the Doll sequences identified herein is defined as the percentage of amino acid residues in a candidate sequence that are identical with the amino acid residues in the Doll sequence, after aligning the sequence and introducing gaps, if necessary, to achieve the maximum percentage sequence identity, and not considering any conservative amino acid substitution as part of the sequence identity. The % identity values used herein can be generated by WU-BLAST-2, which was obtained from (Tatusova TA 1999). WU-BLAST-2 uses several search parameters, most of which are set to the default values.

The term “positive”, in the context of sequence comparison performed as described above, includes residues in the sequence compared that are not identical but have similar properties (e.g. as a result of a conservative substitution). The % value of positive is determined by the fraction of residues scoring a positive value in the BLOSUM 62 matrix divided by the total number of residues in the longer sequence as defined above.

In a similar manner, “percent (%) nucleic acid sequence identity” with respect to the coding sequence of the Doll polypeptides identified herein is defined as the percentage of nucleotide residues in a candidate sequence that are identical with the nucleotide residues in any of the Doll coding sequences of this invention. The identity values used herein can be generated using BLAST module of WU-BLAST-2 set to the default parameters.

The term “epitope tag” refers to a chimeric polypeptide comprising a Doll polypeptide fused to a “tag polypeptide”. The tag polypeptide has enough residues to provide an epitope against which an antibody can be made, yet is short enough that it does not interfere with the activity of the Doll polypeptide to which it is fused.

Nucleic acids are “operably linked” when are placed in a functional relationship with another nucleic acid sequence.

The term “epistasis” means hierarchy in gene action. Epistasis experiments are performed to place components of a signaling pathway in the right order.

The term “rescue experiments” are designed to determine which gene is responsible for a specific mutant phenotype. Specifically, mutant embryos are injected with coding or genomic DNA, and the effect of the introduced DNA is determined on the basis of the capacity to revert the mutant phenotype.

“Active” or “activity” refers to forms of Doll polypeptides that retain the biological and/or immunological activity. A preferred activity includes for instance the ability to modulate the Wnt signaling pathway.

The term “antagonist” is used in a broad sense, and includes any molecule that partially or fully inhibits, blocks or neutralizes a biological activity of Doll polypeptides described herein. In a similar way, the term “agonist” is used in the broadest sense and includes any molecule that mimics or support a biological activity of an active Doll polypeptide.

“Treatment” refers to both therapeutic treatments and prophylactic or preventive measures, wherein the objective is to prevent or slow down the targeted pathologic condition or disorder. Those in need of treatment include those already with the disorder as well as those prone to have the disorder or those in whom the disorder is to be prevented.

### SUMMARY OF THE INVENTION

The present invention relates to a novel family of proteins present in insects and vertebrate organisms, referred to hereinafter as “Daughter of Legless (Doll)” proteins. These proteins play an essential role in the Wnt signaling pathway, and thus in the formation and maintenance of spatial arrangements and proliferation of tissues during development, and in the formation and growth of many human tumors.

In particular, the invention relates to nucleotide sequences of the Drosophila melanogaster doll gene, of proteins encoded by said nucleotide sequences, as well as fragments, derivatives and structural and functional analogs thereof.

In a preferred embodiment the invention relates to the nucleotide and protein sequences of the human and mouse doll homologues, hdoll-1, hdoll-2 and mdoll-1 and mdoll-2, respectively.

In one embodiment, the isolated nucleic acid comprises a sequence encoding a polypeptide having at least 50% amino acid sequence identity, preferably at least about 70% sequence identity, more preferably at least 90% sequence identity, even more preferably at least about 95% sequence identity, yet even more preferably at least about 98% sequence identity, and most preferably 100% identity to (a) a fragment or the entire protein sequence of the Doll polypeptide shown in FIG. 1, or (b) the complement of the nucleic acid molecule coding for (a).

In another preferred embodiment, the isolated nucleic acid encodes a polypeptide having at least 50% amino acid sequence identity, preferably about 70% sequence identity, more preferably at least 90% sequence identity, even more preferably about 95% sequence identity, yet even more preferably about 98% sequence identity, and most preferably 100% identity to (a) a polypeptide which is part or the entire human Doll polypeptides of FIG. 2a/b or (b) the complement of the nucleic acid molecule coding for (a).

In another embodiment, the isolated nucleic acid encodes a polypeptide sequence having at least 50% amino acid sequence identity, preferably about 70% sequence identity, more preferably at least 90% sequence identity, even more preferably about 95% sequence identity, yet even more preferably about 98% sequence identity, and most preferably 100% identity to (a) a polypeptide encoding part of the entire mouse Doll protein of FIG. 3a/b or (b) the complement of the nucleic acid molecule coding for (a).

In a further embodiment, the isolated nucleic acid comprises a sequence encoding a polypeptide with a low overall amino acid sequence identity but shows a sequence identity of at least 60%, preferably at least 70%, more preferably at least 80%, even more preferably at least 90% and most preferably 100% in the conserved domains DHD and PHD (FIG. 4).

In yet another embodiment of the present invention isolated nucleic acids encode polypeptides having a function resembling that of the doll genes.

In another embodiment, the invention relates to a fragment of the Drosophila or vertebrate doll nucleic acid sequences that is applied as hybridization probe. Such nucleic acid fragments are about 20 to about 100 nucleotides in length, preferably from about 20 to about 60 nucleotides in length, most preferably from 20 to 50 nucleotides in length and are derived from the nucleotides sequences shown in FIG. 1, 2 and 3.

The invention further provides eucaryotic and procaryotic expression vectors comprising a nucleic acid molecule encoding Drosophila or vertebrate doll or a fragment thereof as shown in FIGS. 1, 2 and 3. The vector can comprise any of the molecules or fragments thereof described above.

The invention also includes host cells comprising such a vector. By way of example, the host cells can be mammalian cells, yeast cells, insect cells, plant cells or bacteria cells.

Methods of production, isolation and purification of the Doll proteins, derivatives and analogs, e.g. by recombinant means, are also provided (see Example VI, below). In a specific aspect, the invention concerns an isolated Doll peptide, comprising an amino acid sequence of at least 80%, preferably at least about 85% sequence identity, more preferably at least 90% sequence identity, even more preferably at least 95% sequence identity, yet most preferably 100% identity with the amino acid sequences of FIGS. 1, 2 and 3.

In yet another embodiment the invention relates to chimeric proteins comprising a Doll polypeptide fused to a heterologous polypeptide or amino acid sequence. An example of such chimeric molecule comprises a Doll polypeptide fused to an epitope tagged sequence, glutathione-S-transferase protein or to a protein with an enzymatic activity, such as beta-galactosidase or alkaline phosphatase as described in Example VI below.

In a further aspect the invention concerns an isolated full length Doll polypeptide (prepared as described in Example VI), comprising the amino acid sequences of FIG. 1, 2 and 3, or any Doll polypeptide or a fragment thereof described in this invention sufficient to provide a binding site for an anti-Doll anti-body.

In another embodiment the invention provides antibodies that specifically recognize Doll polypeptides. The antibodies can be a polyclonal or a monoclonal preparation or fragments thereof. Polyclonal antibodies are prepared by immunization of rabbits with purified Doll polypeptides prepared as described in Example VI.

The invention also relates to transgenic animals, e.g. Drosophila, mice, rats, chicken, frogs, pigs or sheep, having a transgene, e.g., animals that include and preferably express a heterologous form of the Doll genes described herein, or that misexpress an endogenous or transgenic doll gene. Such a transgenic animal can serve as a model for studying diseases with disrupted Wnt signaling pathway, for the production of Doll proteins, or for drug screening.

In yet another embodiment, the invention also features animals, e.g. Drosophila, mice, rats, chicken, frogs, pigs or sheep, having a mutation in the doll gene, e.g. deletions, point mutations, foreign DNA insertions or inversions. Such animals can serve to study diseases characterized by disrupted Wnt function or in drug screening.

In addition, the invention relates to the use of Doll proteins, homologues, derivatives and fragments thereof as well as nucleic acids, derivatives and fragments thereof in therapeutic and diagnostic methods and compounds. In particular, the invention provides methods and compounds for treatment of disorders of cell fate, differentiation or proliferation by administration of a therapeutic compound of the invention. Such therapeutic compounds include: Drosophila and vertebrate Doll protein homologues or fragments thereof, antibodies or antibody fragments thereto, doll antisense DNA or RNA, doll double stranded RNA, and any chemical or natural occurring compound interfering with Doll function, synthesis or degradation. In particular, the invention provides methods to screen for chemical compounds, organic products or peptides interfering with Doll function in the Wnt pathway. In a preferred embodiment the screening method will be a cellular reporter gene assay or a protein-protein interaction assay.

In another embodiment, a screening assay based on protein-protein interaction is used to screen for compounds specifically inhibiting Doll-Lgs or Doll-interaction partner X.

The invention also provides methods to screen for chemical compounds, organic products or peptides interfering with Doll function in the Wnt pathway.

Furthermore, the invention comprises the use of the DHD domain in screening assays such as an in vitro protein-protein interaction assay or a protein-protein interaction in a host cell. Said assays are applied for the identification of chemical compounds, organic products, polypeptides or peptides interfering with Doll function in the Wnt pathway.

In a preferred embodiment, a therapeutic product according to the invention is administered to treat a cancerous condition or to prevent progression from a pre-neoplastic or non-malignant condition to a neoplastic or malignant state.

In other specific embodiments, a therapeutic product of the invention is administered to treat a blood disease or to promote tissue regeneration and repair. Finally disorders of cell fate, especially hyperproliferative or hypoproliferative disorders, involving aberrant or undesirable expression, or localization, or activity of the Doll protein can be diagnosed by detecting such levels.

### BRIEF DESCRIPTION OF THE DRAWINGS

FIG. 1 The Drosophila doll CDNA and protein sequence.

FIG. 2 The human doll-1 and doll-2 cDNA and protein sequence.

FIG. 3 The mouse doll-1 and doll-2 CDNA and protein sequences.

FIG. 4. Drosophila and human Doll proteins contain a PED finger motif with which they bind to the ED1 of Lgs/BCL9

(A) Top: Schematic representation of Doll, human DOLL-1 and human DOLL-2. The two domains that show high sequence similarities are highlighted in dark gray (DHD: Doll homology domain) and red (PHD: plant homology domain). The DHD appears to be unique, as the inventors failed to find a similar sequence in other Drosophila or human proteins. GenBank accession numbers for Doll, hDoll-1, hDoll-2 are AF457206, AF457207, AF457208, respectively. Bottom: Multiple alignment of Drosophila, human and mouse Doll protein sequences.

(B) Alignment of amino acid sequences of DHD and PHD in Doll and its human homologues.

Similarities are boxed, identities shaded in gray. The numbers to the left indicate the positions of DHD and PHD within their respective protein sequences. For the DHD alignment a gap of 22 aa has been introduced in the Drosophila DHD (represented as (X)5)

(C) Mapping of the Lgs/BCL9 interaction site in Doll. Schematic representation of the proteins tested in the yeast-two-hybrid assay for their interactions with Lgs and BCL9. Results are indicated to the right (“bdg”).

(D) Mapping of the Doll interaction site in digs and hLgs/BCL9. Schematic representation of the proteins tested in the yeast-two-hybrid assay for their interactions with Lgs. The two proteins shown at the bottom were tested by a pull-down assay for both dlgs (numbers without brackets) and hLgs/BCL9 (numbers in parenthesis) with the same result (“bdg”). The deletion removing HD1 comprises aa 318-345 for Lgs and aa 177-204 for hLgs/BCL9. Fusion proteins used were S-Tag-dDoll (aa 542-815) and GST-hDOLL-2 (aa 301-406).

FIG. 5. doll is a segment polarity gene required for Wg signalling

(A-C) Cuticle preparations of larvae derived from wild-type (A), wg mutant (B), and doll mutant embryos (C). The doll¹³⁰/doll¹³⁰ embryo in (C) is derived from a homozygous doll¹³⁰ mutant germ line clone (see Experimental Procedures) and displays a wg-like phenotype.

(D,E) doll functions downstream of dAPC2. Two cuticle preparations are shown from larvae that developed in the absence of maternal and zygotic wild-type dAPC2 function (McCartney et al., 1999). The embryo in (E) additionally lacks the maternal and zygotic function of doll (see Examples). In contrast to dAPC single mutant animals, which have strongly reduced denticle belts, double mutants display a doll-like phenotype.

(F-I) Confocal images of third instar wing imaginal disc preparations stained with antibodies against Doll (F,G) and Ptc (E,I). Wild-type animals show normal expression of these genes (F,H). Discs derived from doll¹³⁰ mutant larvae are small, yet express Ptc (I), but fail to express Dll (H). Lack of Dll expression may be an indirect consequence of the earlier wing-tonotum transformation in doll¹³⁰ larvae. However, we also see a strong reduction of Dll expression in doll¹³⁰ mutant cells from mosaic animals (not shown).

FIG. 6. Lgs and the PHD finger of Doll serve to assemble Doll and Arm

Schematic representation of Lgs (yellow) and Doll (light green) constructs that were used in transgene assays to assess their ability to rescue lgs or doll mutant animals. 1: Full-length Lgs (pOP216, aa 1-1464). 2: C-terminally truncated Lgs (pTK131, aa 1-583). 3: HD1-Gal11-HD2 (pTK153, aa 268-395 (HD1), aa 369-500 (Gal11), aa 465-596 (HD2)). 4: HD1-(HA)3-HD2(pTK143, aa 268-395 (HD1), aa 465-596 (HD2)). 5: Full-length Doll (pTK56, aa 1-815). 6: Doll[DPHD]-HD2 (pTK135, aa 1-740 (Doll[DPHD]), aa 483-561 (HD2)). Transgenes 1 to 4 are able to rescue lgs^{20F} homozygotes. An example for an adult animal rescued by transgene 3 is shown on the right. Transgene 5 can rescue doll¹³⁰ homozygotes. Transgene 6 can rescue doll¹³⁰ as well as lgs^{20F} homozygotes (photographs on the right).

FIG. 7: Rescue of ddoll-/-flies by expression of a human doll transgene. The lethality caused by the doll¹³⁰/EP(3)1076 genotype can be fully rescued by a tubulin 1 promoter-driven transgene that contains either the coding region of the Drosophila doll gene (not shown) or that of one of its two human homologues hdoll-1 and hdoll-2.

FIG. 8: Effects of human Doll 1 and 2 on Tcf transcription. 293 cells were transiently transfected with the pTOPFLASH or pFOPFLASH luciferase reporters and different effector plasmids as indicated. A constitutively active form of β-Catenin (ΔN-β-Catenin, 50 ng) or human Doll-1 or hDoll-2 (350 ng) activate the pTOPFLASH reporter. Cotransfection of human Doll with ΔN-β-catenin strongly enhance the response.

### DETAILED DESCRIPTION OF THE INVENTION

The Wnt signaling cascade is essential for the development of both invertebrates and vertebrates, and has been implicated in tumorigenesis. The Drosophila wg genes are one of the best characterized within the Wnt-protein family, which includes more than hundred genes. In the Drosophila embryo, wg is required for formation of parasegment boundaries and for maintenance of engrailed (en) expression in adjacent cells. The epidermis of embryo defective in wg function shows only a rudimentary segmentation, which is reflected in an abnormal cuticle pattern. While the ventral cuticle of wild type larvae displays denticle belts alternating with naked regions, the cuticle of wg mutant larvae is completely covered with denticles. During imaginal disc development, wg controls dorso-ventral positional information. In the leg disc, wg patters the future leg by the induction of ventral fate (Struhl and Basler 1993). In animals with reduced wg activity, the ventral half of the leg develops into a mirror image of the dorsal side (Baker 1988). Accordingly, reduced wg activity leads to the transformation of wing to notal tissue, hence the name of the gene (Sharma and Chopra 1976). In the eye disc, wg suppresses ommatidial differentiation in favor of head cuticle development, and is involved in establishing the dorso-ventral axis across the eye field (Heberlein, Borod et al. 1998).

Additional genes have been implicated in the secretion, reception or interpretation of the Wg signaling. For instance, genetic studies in Drosophila revealed the involvement of frizzled (Dfz), Dishevelled (dsh), shaggy/zeste-white-3 (sgg/zw-3), armadillo (arm), adenomatous polyposis coli (E-apc), axin, and pangolin (pan) in Wg signaling. The genetic order of these transducers has been established in which Wg acts through Dsh to inhibit Sgg, thus relieving the repression of Arm by Sgg, resulting in the cytoplasmic accumulation of Arm and its translocation to the nucleus. In the nucleus Arm interacts with Pan to activate transcription of target genes. Vertebrate homologues have been identified for all these components (for an updated review see (Peifer and Polakis 2000), suggesting that novel identified members of the Drosophila signaling pathway may likely have vertebrate counterparts.

Mutations leading to nuclear accumulation of the mammalian homologue of Arm, β-Catenin, and consequently to constitutive activation of the Wnt pathway have been observed in many types of cancer, including colon cancer, breast cancer, melanoma, hepatocellular carcinoma, ovarian cancer, endometrial cancer, medulloblastoma pilomatricomas, and prostate cancer (Morin 1999; Polakis, Hart et al. 1999). It is now apparent that deregulation of β-Catenin signaling is an important event in the genesis of these malignancies. However, there are still no known therapeutic agents effectively inhibiting β-Catenin transcriptional activation. This is partly due to the fact that many of the essential components required for its full activation and nuclear translocation are still unknown.

In order to identify new components required for Wingless activation the inventors used a Drosophila genetic approach to screen for dominant suppressors of the rough eye phenotype caused by ectopic expression of Wingless (Wg), the Drosophila homologue of Wnt, during eye development. A new gene, legless (lgs, U.S. Ser. No. 09/915,543) was identified as a strong dominant suppressor of the rough eye phenotype. The gene was subsequently cloned and its in vivo requirement for Wg signal transduction in embryo and in developing tissues was confirmed. The human genome contains at least two human Lgs homologues, hLgs/Bcl9 and hLgs-1. dLgs and hLgs bind to Armadillo and β-Catenin and are functionally required for Wnt signal propagation in invertebrate and vertebrate cells (U.S. Ser. No. 09/915,543). In particular, the presence of Lgs is required for a transcriptional active Arm/Pangolin complex and over-expression of Lgs strongly stimulates the transcriptional output.

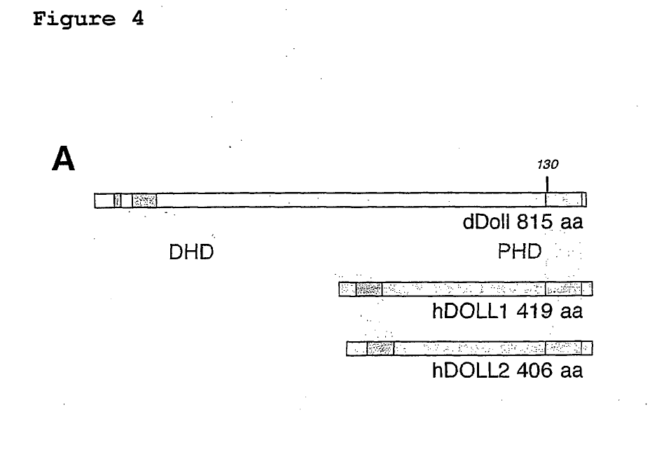

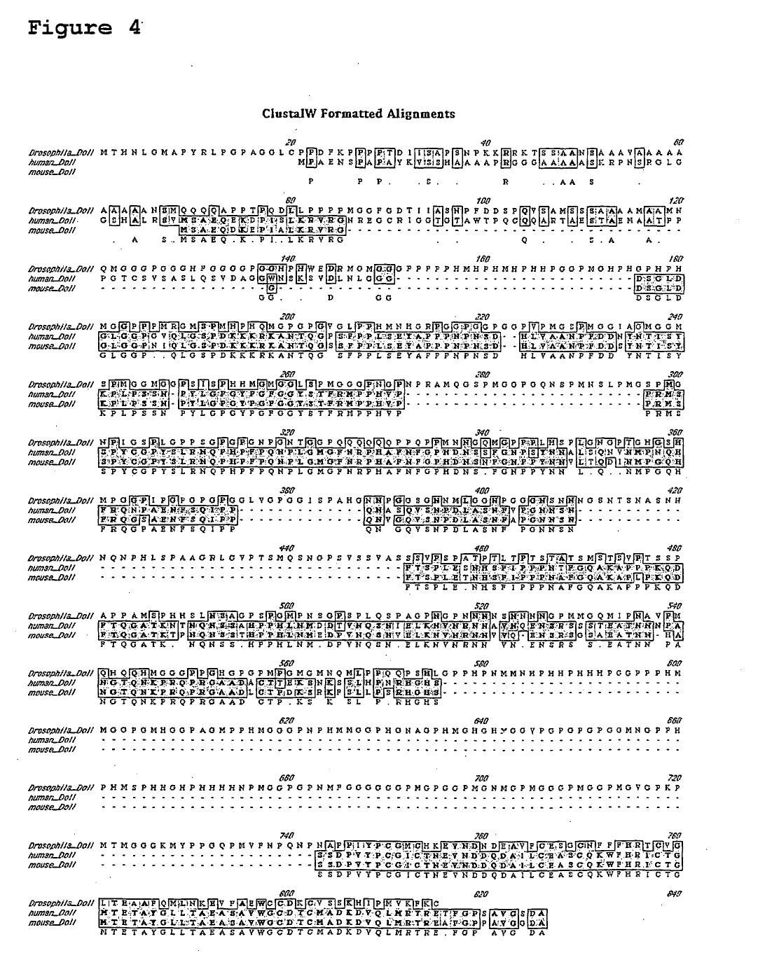

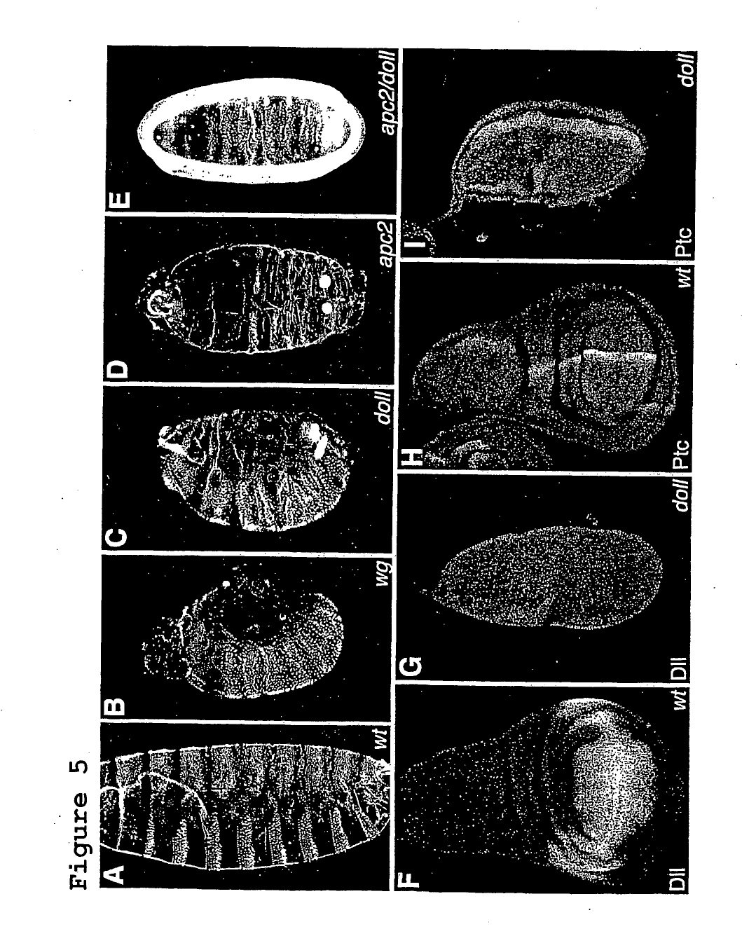

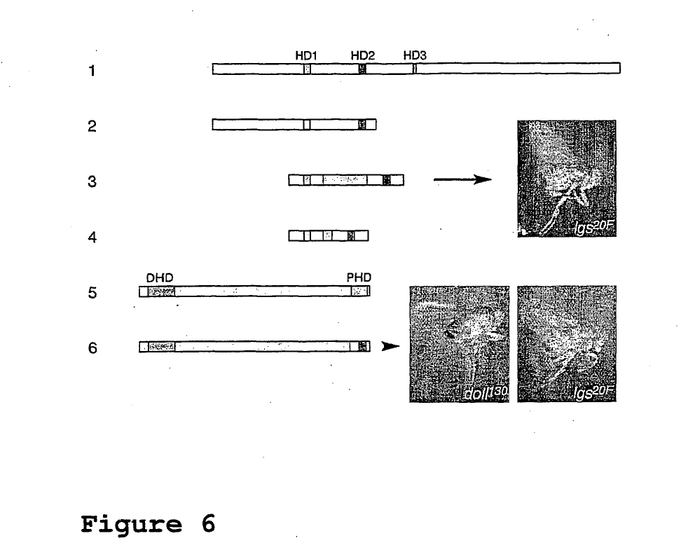

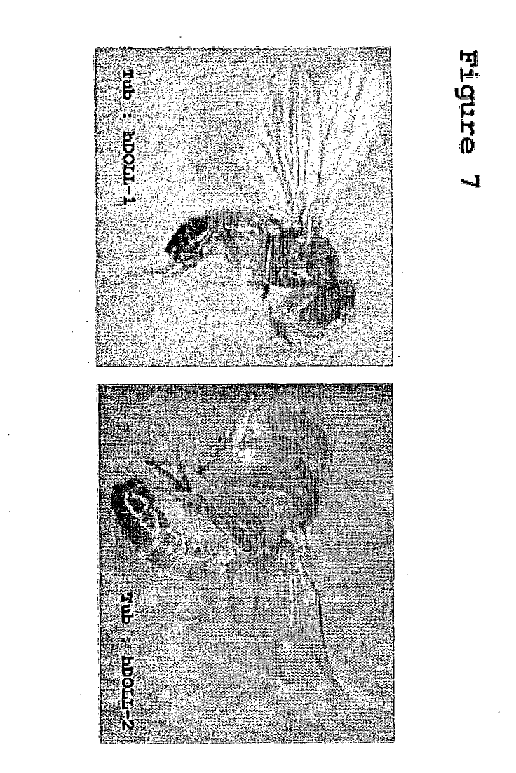

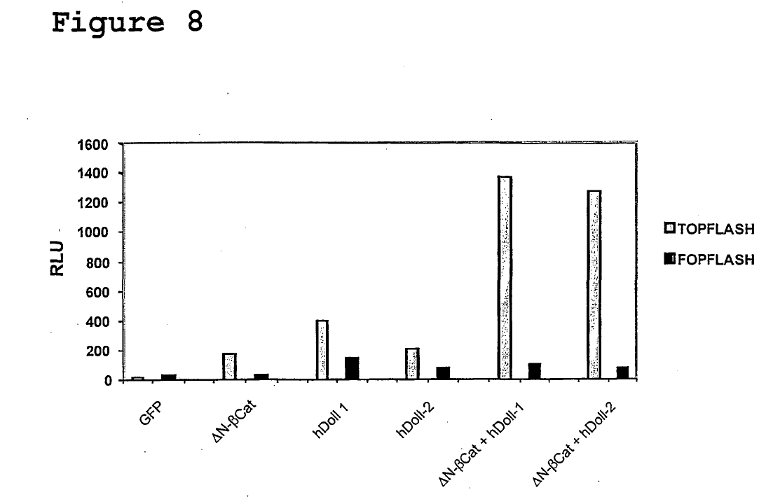

The inventors later made the interesting observation that a mutant form of Lgs protein from which the β-Catenin interacting domain was deleted exhibited a strong dominant-negative effect on Wg-dependent patterning processes when expressed from a transgene in wild-type larvae (data not shown). This strongly suggested that Lgs normally interacts not only with Arm, but also with at least one additional component. In an effort to identify such components yeast-two-hybrid screens for interacting proteins were carried out. In two independent screens in which either the entire protein or the N-terminal half of Lgs was used as a bait, a novel PHD finger protein, referred to as Daughter-of-Legless (Doll), was identified as a Lgs binding protein (FIG. 4). The 815 amino acid residue Doll protein carries a C-terminal domain of 60 amino acids (FIG. 4a), which shows extensive homologies to the PHD (plant homology domain) finger, also known as LAP (leukemia associated protein) domain (Aasland, Gibson et al. 1995). This domain comprises a cysteine rich Zn-binding motif, that has been associated with proteins involved in chromatin-mediated regulation of transcription. The PHD finger of Doll is necessary and sufficient to mediate the interaction to Lgs (FIG. 4c-d). The inventors also demonstrate herein that this interaction is essential for Doll function.

The region of Lgs responsible for Doll-binding was mapped to the HD1 sequence (FIG. 4d). Moreover, two human homologues of the Drosophila doll gene were identified and isolated (FIG. 4a). The protein products of both human genes, hDOLL-1 and hDOLL-2, as well as their mouse homologues possess a highly conserved PHD finger which interacts with the HD1 of hLgs/BCL9 (FIG. 4d). The only other domain in Drosophila Doll, hDoll-1/hDoll-2 and mDoll-1/mDoll-2 that shows significant sequence homology is a 50 amino acid stretch in the N-terminal region, which is referred herein to as ‘Doll homology domain’ (DHD, FIG. 4a,b).

The interaction with Doll appears to be relevant for the in vivo function of Lgs, since a mutant form of Lgs with a deletion of HD1 was unable to rescue lgs mutant animals. The physical association of Doll and Lgs suggested that Doll, like Lgs, may be required for Wg signaling in vivo. To explore this hypothesis a proprietary collection of suppressors of the sev-wg phenotype was searched for mutations that map to the tip of the right arm of chromosome 3, the position of the doll gene. One such suppressor, Sup¹³⁰, mapped to this position, and intriguingly, it showed dominant lethality in combination with the lgs allele lgs^{17E} (U.S. Ser. No. 09/915,543) (Sup¹³⁰/+lgs^{17E}/+transheterozygous animals do not survive). The doll coding region was sequenced using genomic DNA from homozygous Sup¹³⁰ mutant larvae and a 14 bp deletion starting at amino acid position 751 was identified. Hence this allele is referred to as doll¹³⁰ and encodes a truncated Doll protein lacking the C-terminal PHD finger.

The lethality caused by the homozygous dol¹³⁰ genotype can be fully rescued by a tubulin l promoter-driven transgene that contains either the coding region of the Drosophila doll gene or that of one of its two human homologues hDoll-1 and hDoll-2 (FIG. 7). Thus, the vertebrate homologues of doll were confirmed genetically to be true functional homologues of Doll, and hence the vertebrate homoiogues are part of this invention. To assay the possible role of Doll in Wg signal transduction during development, embryos homozygous for the doll¹³⁰ mutation that derived from female germ cells equally mutant for doll were generated. Doll mRNA is maternally contributed and strongly and ubiquitously expressed during all the developmental stages. Consequently, only embryos lacking both embryonal and maternal doll are characterized by a severe segment polarity phenotype (FIG. 5A-C), while weaker loss of function doll mutants display pupal lethality with a partial or complete loss of the antennae and the legs. Mutant individuals lacking only zygotic function survive until early pupal stages and exhibit imaginal discs that are abnormally small. The Hh target gene ptc was expressed at wild-type levels in these discs, however, no expression of the Wg target Dll could be detected (FIG. 5F-I). These discs appear to lack the presumptive wing blade field and possess two primordia for the notum (FIGS. 5G and I). The fact that similar phenotypes are caused by loss of function of wg, dsh, arm, or lgs confirms the essential role of doll in the Wg signaling pathway.

To address the role of Doll in β-Catenin-mediated transcription a TCF reporter gene (TOPFLASH, (Morin, Sparks et al. 1997)) was used in immortalized human embryo kidney cells (HEK 293 cells). Low levels of a stable mutant form of β-Catenin (ΔN-β-catenin; (van de Wetering, Cavallo et al. 1997)) were introduced into these cells to partially stimulate the pathway. The additional expression of hDoll-1 (FIG. 8) or hDoll-2 (not shown) lead to a large increase in luciferase activity (30-fold). These levels are significantly higher than the sum of those produced by either treatment alone (FIG. 8). This potentiation of β-Catenin activity by hDoll-1 and 2 appears to be mediated by the interaction of endogenous TCF protein with its DNA target sites, as it is only observed with TOPFLASH, which contains five optimal TCF binding sites, but not with the control reporter FOPFLASH, which contains five mutated sites (Morin, Sparks et al. 1997). Thus this experiment adds supportive evidence to the notion that Doll proteins transduce Wnt signals by activating TCF target genes in a β-Catenin-dependent manner.

In summary, the protein-protein interactions demonstrated between Drosophila Doll and Lgs and those between their human homologues human Doll and hLgs/Bcl9, respectively, in conjunction with the genetic and cell biological data show that Doll proteins are positive regulators of the Wg and Wnt signaling pathways, respectively.

### EXAMPLES

### Example I

### Isolation of Doll cDNA

The cDNA for Daughter of Legless (Doll) was isolated in two independent yeast genetic screens of a Drosophila cDNA-library for proteins directly binding to Lgs. Other DNA libraries can be used as well, such a genomic and CDNA libraries from vertebrate and invertebrate organisms. Other methods than a yeast-two hybrid screen can be used as well. Such methods include, but are not limited to, direct amplification using gene specific primers and standard methods known by people skill in the art. To perform the yeast two-hybrid screening for protein binding to Lgs CDNA sequences encoding the first 732 amino acids (“LgsN”) and the full-length protein of 1464 amino acids (“LgsFL”) were subcloned into a yeast expression vector (pLexA, Clontech), fusing them to the LexA DNA-binding domain. Subsequently these constructs were transformed into the LEU2-reporter yeast strain EGY48 together with the lacZ-reporter plasmid pSH18-34 and an embryonic Drosohila melanogaster cDNA-library fused to an acidic transcriptional activation domain (“RFLY-1” library, PNAS 93, 3011-3015). In a first step triple-transformant colonies containing the LgsN- or LgsFL-LexA-fusion constructs, respectively, the pSH18-34 reporter and a RFLY-1 library plasmid were grown on minimal selective medium plates for two days, harvested, thoroughly mixed, and stored as uniform aliquots. Then cells from one of these aliquots were transferred into permissive Galactose/Raffinose minimal selective liquid medium, and incubated with shaking at 30° C. for a few hours, thereby inducing expression of the library cDNA-activation domain fusion from the GAL1-inducible promotor. Finally these “induced” cells were plated on Galactose/Raffinose minimal selective medium plates lacking the amino acid 1-leucine. On these plates cell growth was sustained only upon activation of a LEU2-selector gene through molecular interaction of the respective LexA-fusion and activation domain-fusion proteins. The LEU2-gene codes for an essential metabolic enzyme needed for the biosynthesis of leucine from other amino acid precursors. All clones growing under these restrictive conditions were isolated and analyzed for the activity of the lacZ-reporter gene, encoding the metabolic enzyme β-Galactosidase from the enterobacterium E.coli, by a standard X-Gal assay (e.g. Bartel and Fields (eds.), Oxford University Press 1997). From all candidate clones that passed these two selection steps, the cDNA-library plasmids were isolated again by standard techniques (e.g. Methods in Yeast Genetics, Cold Spring Harbour Laboratory Press, 1997) and retested for specific interaction with Lgs in the X-Gal assay, using an unrelated LexA-fusion protein as a negative control. By this procedure three independent cDNA-clones were identified, that strongly and specifically interacted only with Lgs and contained partially overlapping sequences: BK12b, BK14b and TK5.35h. By searching the Drosophila genome database using the blastn algorithm (http://www.ncbi.nlm.nih.gov:80/BLAST/) we mapped the three isolated cDNAs to the CG11518 locus, coding for a protein product of 815 amino acids in length. The cDNA-clones coded different parts of the Doll protein, with BK12b containing nucleotides (nt) 2223-2448, BK14b nt 2191-2448 and TK4.35h nt 749-2448 of the computationally predicted open reading frame (ORF). Further bioinformatical analysis (http://www.ebi.ac.uk/interpro/) revealed that the very C-terminal part of the protein sequence (ca. aa 745-805), present in all three of the Lgs-binding clones, was predicted to adapt a PHD-finger fold, which has been identified in other proteins involved in transcriptional regulation at different levels.

### Example II

### Identification of Human and Mouse Homologues of Drosophila Doll

After the identification of the Drosophila Doll amino acid sequence, publicly available databases were searched for similar protein sequences in other species, using the tblastn algorithm (http://www.ncbi.nlm.nih.gov:80/BLAST/). Two candidate sequences were found each in ESTs from Mus musculus and Homo sapiens, respectively, the putative protein products of which display high similarity to Doll in their C-terminal domains. These stretches of high similarity are predicted to adapt a PHD-finger fold as well (FIG. 4). Doll proteins do not display other known structural motifs in their N-terminal sequences but they display a second high homology domain, which was accordingly named DOLL Homology Domain (bHD) (FIG. 4). Doll proteins of both invertebrate and vertebrate origin have so far not been further described or experimentally studied, and have thus not previously been implicated in any specific biological process.

### Example III

### Isolation and Mapping of Drosophila Doll Alleles

EMS-treated males were crossed to females carrying a wg transgene (sev-wg) driven by two copies of the sevenless enhancer (Basler, Christen et al. 1991). 2×105 progeny were screened for suppressors of the rough eye phenotype. Third chromosomal suppressors were coarsely mapped by meiotic recombination using a panel of P[y+] insertions. One such suppressor, Sup¹³⁰, showed intriguingly dominant lethality in combination with the lgs allele lgs^{17E} (U.S. Ser. No. 09/915,543) (Sup¹³⁰/+lgs^{17E}/+transheterozygous animals do not survive), strongly suggesting a close genetic interaction. Fine mapping of the mutation using denaturing HPLC (WAWE system, Transgenomic Inc.) demonstrated that it localizes within the doll gene. The doll coding region was therefore sequenced using PCR fragments covering the doll coding region derived from genomic DNA from homozygous Sup¹³⁰ mutant larvae. The defect in Sup¹³⁰ was found to be a 14 bp deletion (nucleotides 2253 to 2266: 5′ CATGTGCCACAAGG 3′) within the doll open reading frame that induced a frame-shift subsequent to amino acid 751 and resulted in the formation of a premature stop codon. Hence this allele is referred to as doll¹³⁰ and encodes a truncated Doll protein lacking the C-terminal PHD finger.

Pole cell transplantation, chromosome squashes, and chromosome in situ hybridization experiments were carried out according to standard protocols (Ashburner 1989).

### Example IV

### Use of Doll as a Hybridization Probe

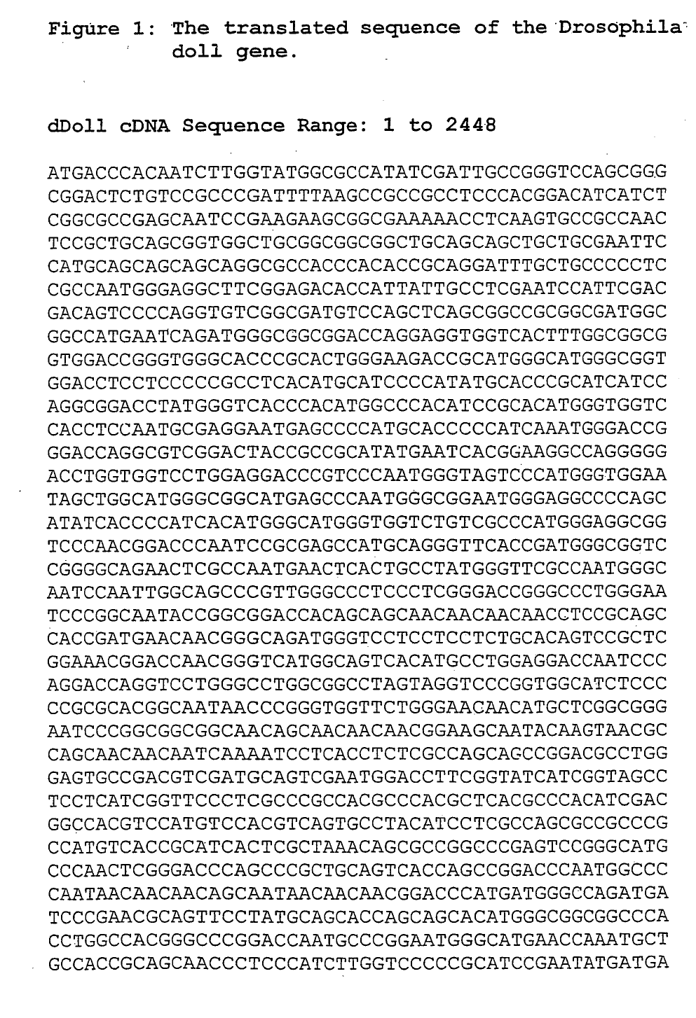

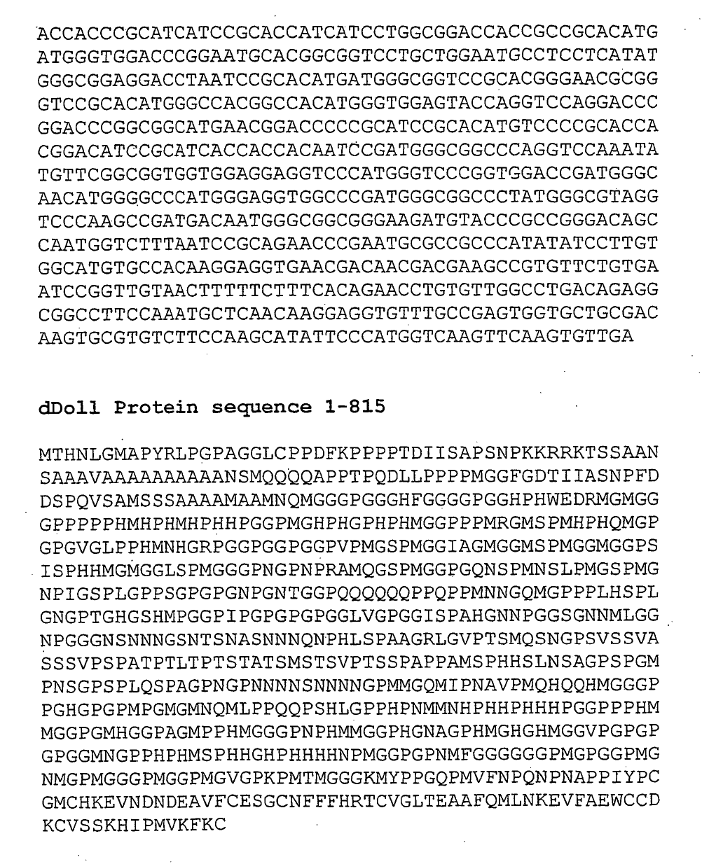

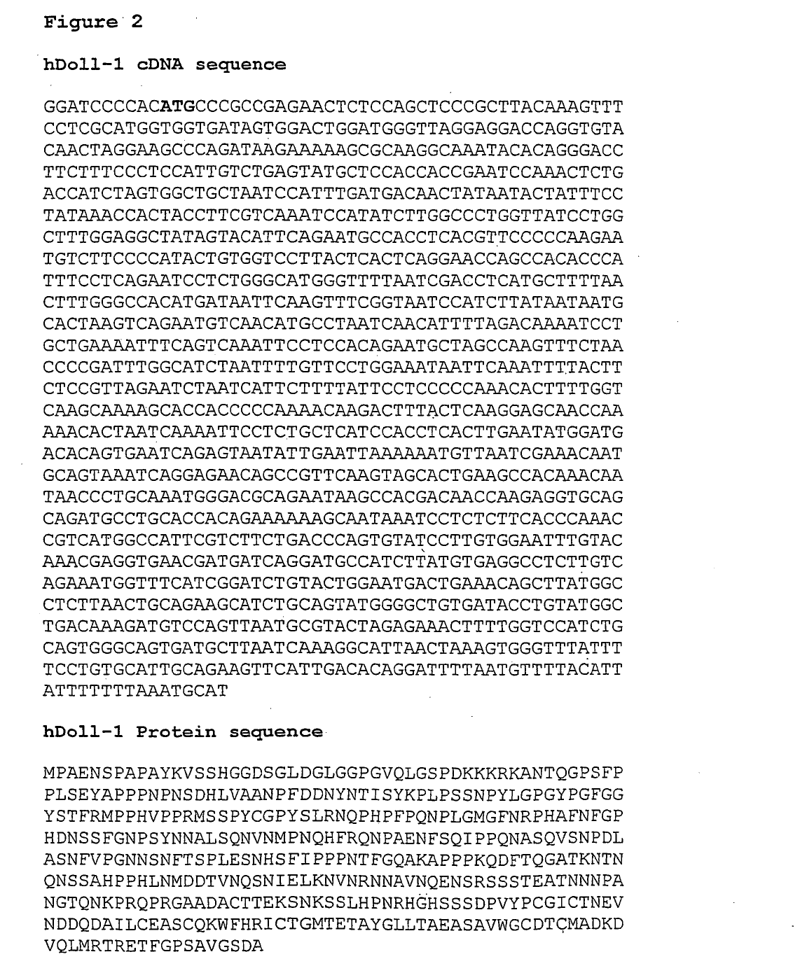

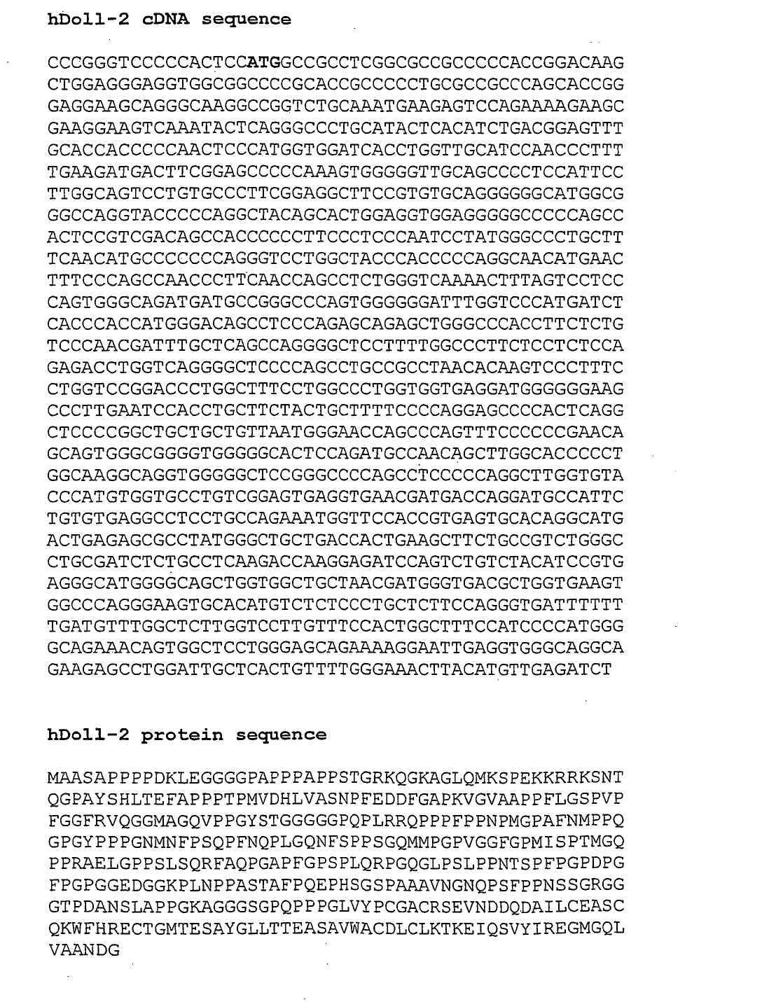

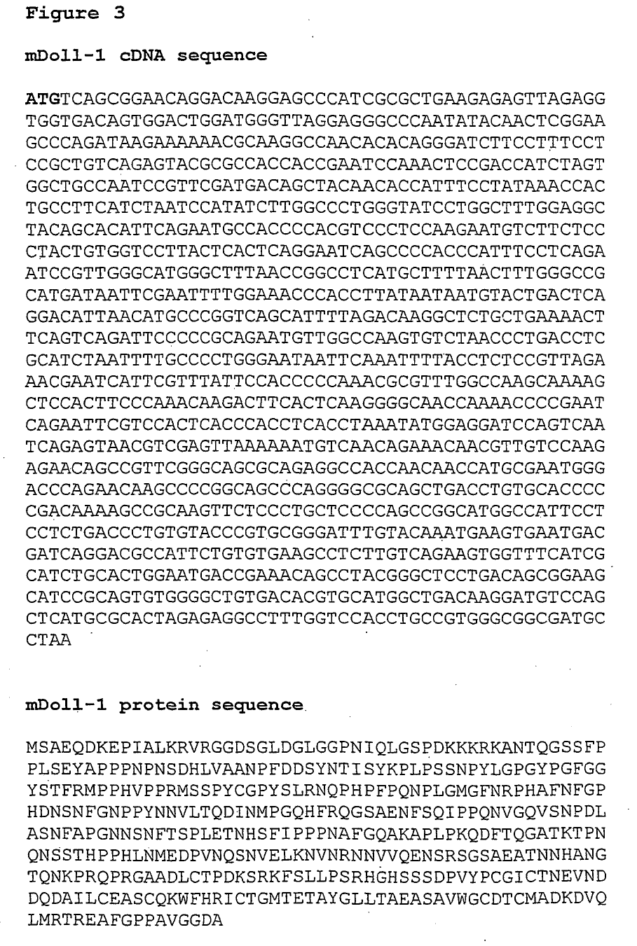

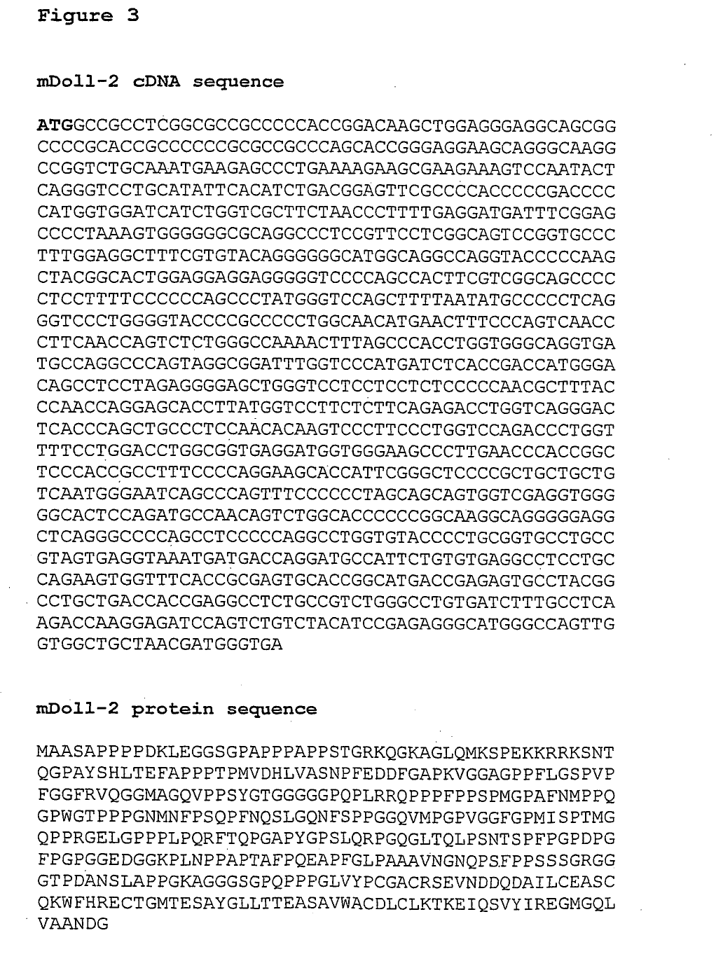

The following method describes the use of a non-repetitive nucleotide sequence of doll as a hybridization probe. The method can be applied to screen for doll homologues in other organisms as well. DNA comprising the sequence of doll (as shown in FIGS. 1,2,3) is employed as probe to screen for homologue DNAs (such as those included in CDNA or genomic libraries). Hybridization and washing of the filters containing either library DNAs is performed under standard high stringency conditions (Sambrook, Fritsch et al. 1989). Positive clones can be used to further screen larger cDNA library platings. Representative cDNA-clones are subsequently cloned into pBluescript (Stratagene) or similar cloning vectors and sequenced.

### Example V

### Use of Doll as a Hybridization Probe for in situ Hybridization

In situ hybridization of Drosophila doll mRNA can be performed in embryo as described in (Tautz and Pfeifle 1989). However, with small modifications it can also be used to detect any mRNA transcript in Drosophila larval imaginal discs or vertebrate tissue sections. Labeled RNA probes can be prepared from linearized doll CDNA (as showed in FIGS. 1,2,3), or a fragment thereof, using the DIG RNA labeling Kit (SP6/T7) (Boehringer Mannheim) following the manufacturer's recommendations.

### Example VI

### Expression of Doll in Drosophila melanogaster

Doll can be expressed in Drosophila in the whole organism, in a specific organ or in a specific cell type, during the whole life or only at a specific developmental stage, and at different levels. An overview of the standard methods used in Drosophila genetics can be found in (Brand and Perrimon 1993; Perrimon 1998; Perrimon 1998).

Generation of Doll Mutant Embryos

Mosaic germlines are generated with the help of site-specific recombination through the FLP recombinase (Xu and Rubin 1993).

Females of the genotype hsp70:flp, FRT82 doll¹³⁰/FRT82 ubi-GFP are heat-shocked at 37° C. for 1 hr during the third instar larval stage to induce FLβ-directed recombination and later mated to doll¹³⁰/TM6b[y+] males. Germline mosaics are induced. The source of recombinase is a first chromosome insertion of a fusion of the hsp 70 promoter (denoted by “hsp70”) to the FLP coding sequence. Somatic recombination at the FRT82 sites gives rise to adult female germ line that produces oocytes that upon fertilization lead to embryos which do not contain neither zygotic nor maternally contributed information for the production of functional dDoll protein. Those embryos can be identified by the absence of the yellow+phenotype provided by the TM6b[y+] paternal balancer chromosome. For analysis, cuticles are prepared by standard techniques from mutant embryos, and examined by dark field microscopy.

dApC2 doll doublemutant germ line clones were generated with an FRT82 dAPC2 DS doll¹³⁰ chromosome. The FRT82 ovo^{D1 chromosome (Chou and Perrimon} 1996) was used to select for mutant germ cells. The FRT82 doll¹³³ chromosome was also used to create doll mutant clones in discs, in conjunction with an FRT82 arm-lacz chromosome.

Generation of Doll Mutant Embryos Expressing Constitutively Active Arm

In order to express constitutively active Arm (“ΔArm”),h females of the genotype described above are heat shocked at 37° C. for 1 hr during late pupal stages and mated to males of the genotype UAS:ΔArm hsp70-Gal4/UAS:ΔArm hsp70-Gal4; doll/TM6b[y+]. Due to the presence of the additional transgenes in these males off-spring that had arisen from a doll mutant oocytes and doll mutant sperm express upon heat treatment the constitutively active Arm protein, that transiently induced Wingless target genes.

### Example VII

### Rescue of Ddoll-/-flies with Hdoll-1 and Hdoll-2 cDNA Expression

In order to confirm the functional homology between Drosophila and human Doll-1 and human Doll-2, the human genes were introduced into Drosophila flies carrying two mutant doll alleles (ddoll-/-flies) Specifically, flies carrying e.g. a tub:hdoll transgene, and two mutant doll alleles, e.g. doll¹³⁰ and EP(3)1076 (publicly available) were generated. ddoll-/-mutant flies display larval or pupal lethality. In contrast, ddoll-/-mutant flies carrying at least one copy of the tub:hdoll-1 or tub:hdoll-2 transgenes survive to adulthood. This demonstrates that both, hDoll-1 and hDoll-2, can replace endogenous ddoll function in flies and thus validates functional homology between Drosophila and human Doll (FIG. 7).

### Example VIII

### Protein Production and Purification of Doll in E. coli

The following method describes recombinant expression of Doll proteins in bacterial cells. DNA encoding full-length or a truncated Doll form is fused e.g. downstream of an epitope tag or glutathione-S-transferase (GST) cDNA and a thrombin or enterokinase cleavage site contained within an inducible bacterial expression vector. Such epitope tags include poly-his, S-protein, thioredoxin and immunoglobin tags. A variety of plasmids can be employed, including commercially available plasmid such as pGEX-4T (Pharmacia) or pET-32a (Novagen).

Briefly, a bacterial expression plasmid containing the doll sequence, for instance fused to a GST-sequence, is transformed by conventional methods into protease deficient E.coli such as BL21 (Stratagene). A bacterial colony containing the plasmid is then expanded overnight in selection medium to reach saturation. The next morning, this culture is diluted 1:100 and bacterial are allowed to growth to an optical density (OD₆₀₀) of 0.6. Protein production is initiated by addition of an inducer of the promoter under which GST-Doll fusion protein is expressed. A variety of inducers can be employed depending on the expression vector used, including IPTG.

Expressed GST tagged Doll can then be purified, for instance, using affinity beads or affinity chromatography, such as glutathione beads (commercially available from Pharmacia). Extracts are prepared by lysing the Doll-expressing bacteria in sonication buffer (10 mM Tris HCl pH 8.0, 150 mM NaCl, 1 mM EDTA, 1.5% sarkosyl, 2% Triton-X-100, 1 mM DTT and protease inhibitors), followed by short sonication on ice (e.g. 3 times 20 seconds at middle power) and centrifugation. Cleared supernatants are then incubated under gentle rotation for example with glutathione beads for 1 hrs at 4° C. Next beads are washed several time in washing buffer (20 mM Tris pH 8.0, 200 mM NaCl, 1 mM EDTA, 1 mM DTT, 1 mM MgCl₂, 0.5% NP40), and finally stored in storage buffer (20 mM Tris pH 8.0, 200 mM NaCl, 1 mM EDTA, 1 mM DTT, 1 mM MgCl2, 10% glycerol, 0.5% NP40, and proteinase inhibitors).

Alternatively, a His-tagged, S-protein, thioredoxin or IgG tagged Doll can be purified using affinity chromatography.

The quality of the preparations can be checked e.g. by SDS-gel electrophoresis and silver staining or Western blot.

In case the epitope-tag has to be cleaved, several methods are available depending on the presence of a cleavage site between the epitope-tag and the Doll protein. For example, it is possible to produce a GST-Doll fusion protein containing a thrombin cleavage site right before the first Doll amino acid. Briefly, a GST-Doll preparation on glutathione-affinity beads is washed several times in cleavage buffer (50 mM Tris HCl pH 7.0, 150 mM NaCl, 1 mM EDTA, 1 mM DTT). Thrombin is then added and the samples are incubated for over 16 h at room temperature. Supernatants are then collected and analyzed for successful cleavage of Doll from the beads by polyacrylamide gel electrophoresis and silver staining or Western blot.

### Example IX

### Protein-protein Interactions Involving Doll

A GST-fusion protein in vitro binding assay can be performed to map binding domains and find additional interaction partners. For this purpose, proteins are in vitro translated using reticulocyte lysates (e.g. TNT-lysates, Promega Corporation) containing [3⁵S] methionine following the instructions provided by the manufacturer. Alternatively, cellular proteins can be labeled by incubation of culture cells with [³⁵S]methionine. Glutathione Stransferase (GST) fusion proteins, produced as illustrated in the Example VIII, are immobilized on glutathione-Sepharose and blocked in binding buffer (20 mM Tris pH 8.0, 200 mM NaCl, 1 mM EDTA, 1 mM DTT, 1 mM MgCl², 10% glycerol, 0.5% NP40, 0.05% BSA, and proteinase inhibitors) for 45 min. Two μg of immobilized GST proteins are then incubated for 1.5 hrs with 0.5-6 μl of in vitro translated proteins in binding buffer or with [³⁵S]methionine labeled cell extract. The beads are washed four times in washing buffer (20 mM Tris pH 8.0, 200 mM NaCl, 1 mM EDTA, 1 mM DTT, 1 mM MgCl₂, 0.5% NP40) and boiled in Laemmli SDS sample buffer. Proteins binding to Doll are detected by autoradiography. In case that a cell lysate were used to identify novel Doll binding partner, the protein bands on the gel can be isolated by methods known in the art, and the protein sequence can be determined e.g. by mass spectrophotometrical analysis.

A yeast two hybrid assay can additionally be performed to confirm the results of the in vitro binding assays described above or to screen cDNA library for new interaction partners (Fields and Sternglanz 1994). In this context, the desired cDNAs are subcloned into appropriate yeast expression vectors that link them either to a Lex DNA binding domain (e.g. pLexA, Clontech) or an acidic activation domain (e.g. pGJ4-5, Clontech). The appropriate pair of plasmids is then transformed together with a reporter plasmid (e.g. pSH18-34, Clontech) into an appropriate yeast strain (e.g. EGY48) by the lithium acetate-polyethylene glycol method and grown on selective media (Sambrook, Fritsch et al. 1989). Transformants are analyzed for reporter gene activity as described by the manufacturer of the vector-reporter plasmid used. To establish reproducibility the interactions is tested in both directions. Alternatively, this method is used to screen for novel Doll interaction partners. In this context, e.g. pLexA-Doll is transfected into yeast together with a cDNA library cloned into e.g. pGJ4-5 as described above. Positive clones can be isolated and the cDNA they contain can be sequenced by methods known by people skilled in the art.

### Example X

### Immunohistochemistry

Localization of the Doll proteins is performed on Drosophila embryo, imaginal discs, invertebrate and vertebrate adult tissue sections or tumor cell lines using the anti-Doll antibodies provided by this invention. For instance, if a tumor cell line is used, cells can be seeded into polylysine-coated 8 well chambers (Nalge-Nunc Internat.) and grown overnight at 37° C. As a positive control, 293 MEK cells (ATCC) cells might be transfected e.g. by a lipofection method (e.g. Lipofectamine, Gibco technologies) with a Doll expression plasmid, such as pcDNA3.1 (Invitrogen). Two days after transfection, cells are washed and fixed with 3.7% formaldehyde in PBS for 10 min, permeabilized in 0.5% Triton-X-100 for another 10 min, and blocked with a 1:1000 dilution of pre-immunoserum in 2% BSA-PBS for 1 h at RT. Cells are then incubated with a 1:1000 dilution of anti-Doll immunoserum for 2 hrs at RT, followed by washing in PBS and staining with anti-rabbit secondary-antibody. The washing step is repeated and preparations are blocked in a solution of 3% BSA in PBS/0.1% TritonX-100 for 1 hr. The slides are then washed three times for 5 min in PBS and incubated with a 1:200 dilution (v/v) of TRITC-conjugated swine anti-rabbit immunoglobulin (Dako, Inc.). The washing step is repeated before applying coverslips using Vectashield® mounting medium (Vector Laboratories, Inc.). Detection of other proteins such as β-Catenin, hLgs or Tcf can be performed in the same way using anti-β-Catenin (commercially available), anti-hLgs (U.S. Ser. No. 09/915,543) or anti-Tcf (commercially available) specific antibodies, respectively.

### Example XI

### Luciferase Reporter Gene Assays

The effect of Doll on Tcf transactivation activity can be performed in a cell culture system using a Tcf responsive luciferase reporter gene. Depending on the expression vector used, this protocol can be applied for mammalian as well as for Drosophila cell lines. For instance, HEK293 cells (ATCC) are a well suitable system. Hereby, Doll full length cDNA is cloned into a mammalian expression vector, such as pcDNA3 (Invitrogen), and transfected together with the TOPFLASH luciferase reporter plasmid (Upstate biotechnology, New York, USA) into 293 cells. A lipofection agent like the Lipofectamine transfection reagent (Life Technologies, Inc.) can be used for this purpose. A renilla luciferase reporter plasmid, e.g. pRL-SV40, (Promega Corporation, Madison USA), is co-transfected to normalize the transfection efficiency. Cell extracts are prepared 48 h after transfection and assayed for firefly and renilla luciferase activity as described by the manufacturer (Dual luciferase reporter assay system, Promega Corporation). All the luciferase values are normalized for renilla luciferase activity (see FIG. 8).

### Example XII

### Screening of Chemical Compounds, Organic Products or Peptides Interfering with Doll Function

A reporter gene assay is performed with a similar protocol as described in example XI, but scaled down to be performed as a high throughput screening. For this purpose colon cancer cell lines with mutated and/or constitutively active β-Catenin are stably transfected with the Topflash vector described in Example XI and Doll cDNA. The established monoclonal population, which gives the most reliable and constant reporter gene activity is selected for later assays. One day after plating, cells are treated with single compounds derived from a chemical or peptide library. One to 24 hours later reporter gene activity is measured. Compounds found to inhibit reporter gene activity are then further characterized for specific activity on the Doll-containing transcriptional complex. Alternatively, Wnt pathway activity can be measured by detecting mRNA or protein levels of a target gene, e.g. myc (He, Sparks et al. 1998).

### Example XIII

### Screening Assay Based on Protein-protein Interaction for Compounds Inhibiting Doll-Lgs or Doll Interaction Partner X

Doll and its interaction partner or fragments thereof are produced and purified e.g. from E.coli cultures (e.g. as described in example VIII). Proteins are tagged e.g. with 6 histidines, Sprotein, GST or thioredoxin. Small aliquots of the purified proteins are incubated in an appropriate binding buffer. At this point chemical compounds are added to the mixture and their capacity to disrupt the protein-protein interaction is monitored e.g. by any of the methods described below. Compounds inhibiting this interaction are subsequently tested for their specificity and in vivo toxicity. Well established methods to monitor protein-protein interactions are e.g.:

Time resolved fluorometry with lanthanide chelate labels (Hemmilä I. And Webb S. DDT 2: 373-381 (1997))

Scintillation proximity assay (SPA) (Amersham life Science)

Fluorescence polarisation

### References

Aasland, R., T. J. Gibson, et al. (1995). “The PHD finger: implications for chromatin-mediated transcriptional regulation.” Trends Biochem Sci 20(2): 56-9.

Ashburner, M. (1989). Drosophila A laboratory handbook. Cold Sring Harbor, N.Y., Cold Spring Harbor Laboratory Press.

Baker, N. E. (1988). “Transcription of the segment-polarity gene wingless in the imaginal discs of Drosophila, and the phenotype of a pupal-lethal wg mutation.” Development 102(3): 489-97.

Barker N, H. G., Korinek V, Clevers H (1999). “Restricted high level expression of Tcf-4 protein in intestinal and mammary gland epithelium.” Am J Pathol 154: 29-35.

Basler, K., B. Christen, et al. (1991). “Ligand-independent activation of the sevenless receptor tyrosine kinase changes the fate of cells in the developing Drosophila eye.” Cell 64(6): 1069-81.

Brand, A. H. and N. Perrimon (1993). “Targeted gene expression as a means of altering cell fates and generating dominant phenotypes.” Development 118(2): 401-15.

Cabrera, C. V., M. C. Alonso, et al. (1987). “Phenocopies induced with antisense RNA identify the wingless gene.” Cell 50(4): 659-63.

Chou, T. B. and N. Perrimon (1996). “The autosomal FLP-DFS technique for generating germline mosaics in Drosophila melanogaster.” Genetics 144(4): 1673-9.

Fields, S. and R. Sternglanz (1994). “The two-hybrid system: an assay for protein-protein interactions.” Trends Genet 10(8): 286-92.

Grosschedl R, E. Q. (1999). “Regulation of LEF-1TCF transcription factors by Wnt and other signals.” Current Opinion in Cell Biology 11: 233-240.

He, T. C., A. B. Sparks, et al. (1998). “Identification of c-MYC as a target of the APC pathway [see comments].” Science 281(5382): 1509-12.

Heberlein, U., E. R. Borod, et al. (1998). “Dorsoventral patterning in the Drosophila retina by wingless.” Development 125(4): 567-77.

Morin, P. J. (1999). “beta-catenin signaling and cancer.” Bioessays 21(12): 1021-30.

Morin, P. J., A. B. Sparks, et al. (1997). “Activation of betacatenin-Tcf signaling in colon cancer by mutations in betacatenin or APC [see comments].” Science 275(5307): 1787-90.

Nusse, R. and H. E. Varmus (1982). “Many tumors induced by the mouse mammary tumor virus contain a provirus integrated in the same region of the host genome.” Cell 31(1): 99-109.

Peifer, M. and P. Polakis (2000). “Wnt signaling in oncogenesis and embryogenesis—a look outside the nucleus.” Science 287(5458): 1606-9.

Perrimon, N. (1998). “Creating mosaics in Drosophila.” Int j Dev Biol 42(3): 243-7.

Perrimon, N. (1998). “New advances in Drosophila provide opportunities to study gene functions.” Proc Natl Acad Sci USA 95(17): 9716-7.

Perrimon, N. and A. P. Mahowald (1987). “Multiple functions of segment polarity genes in Drosophila.” Dev Biol 119(2): 587-600.

Polakis, P., M. Hart, et al. (1999). “Defects in the regulation of beta-catenin in colorectal cancer.” Adv Exp Med Biol 470: 23-32.

Potter, J. D. (1999). “Colorectal cancer: molecules and populations.” Journal of the National Cancer Institute 91(11): 916-32.

Rijsewijk, F., M. Schuermann, et al. (1987). “The Drosophila homolog of the mouse mammary oncogene int-1 is identical to the segment polarity gene wingless.” Cell 50(4): 649-57.

Roose, J. and H. Clevers (1999). “TCF transcription factors: molecular switches in carcinogenesis.” Biochimica et Biophysica Acta 1424 (2-3): M23-37.

Sambrook, J., E. F. Fritsch, et al. (1989). Molecular cloning: a laboratory manual, Cold Spring Harbor Laboratory Press.

Sharma, R. P. and V. L. Chopra (1976). “Effect of the Wingless (wg1) mutation on wing and haltere development in Drosophila melanogaster.” Dev Biol 48(2): 461-5.

Struhl, G. and K. Basler (1993). “Organizing activity of wingless protein in Drosophila.” Cell 72(4): 527-40.

Tatusova TA, M. T. (1999). “Blast 2 sequences—a new tool for comparing protein and nucleotide sequences.” FEMS Microbiol Lett. 174: 247-250.

Tautz, D. and C. Pfeifle (1989). “A non-radioactive in situ hybridization method for the localization of specific RNAs in Drosophila embryos reveals translational control of the segmentation gene hunchback.” Chromosoma 98(2): 81-5.

van de Wetering, M., R. Cavallo, et al. (1997). “Armadillo coactivates transcription driven by the product of the Drosophila segment polarity gene dTCF.” Cell 88(6): 789-99.

Waltzer, L. and M. Bienz (1999). “The control of beta-catenin and TCF during embryonic development and cancer.” Cancer & Metastasis Reviews 18(2): 231-46.

Willis, T. G., I. R. Zalcberg, et al. (1998). “Molecular cloning of translocation t(1;14) (q21;q32) defines a novel gene (BCL9) at chromosome lq21.” Blood 91(6): 1873-81.

Wodarz, A. and R. Nusse (1998). “Mechanisms of Wnt signaling in development.” Annual Review of Cell & Developmental Biology 14: 59-88.

Xu, T. and G. M. Rubin (1993). “Analysis of genetic mosaics in developing and adult Drosophila tissues.” Development 117(4): 1223-37.

1. A nucleic acid sequence coding for a polypeptide being part of at least one signaling pathway in insects and vertebrates, characterized in that said nucleotide sequence is the “Daughter of Legless” (DOLL) gene as well as homologues, fragments, derivatives and functional and structural analogs thereof.

2. The nucleic acid sequence according to claim 1, characterized in that said signaling pathway is the Wnt signaling pathway.

3. The nucleic acid sequence according to claim 2, characterized in that it is the Drosophila melanogaster doll gene (ddoll) comprising the nucleotide sequence as shown in SEQ. ID. NO 1.

4. The nucleic acid sequence according to claim 3, characterized in that it is coding for a polypeptide comprising 50% to 100%, preferably 100% sequence identity to a fragment or the entire sequence of the Drosophila Doll polypeptide as shown in SEQ. ID. NO 6.

5. The nucleic acid sequence according to claim 2, characterized in that it is the human doll-1 (hdoll-1) comprising the nucleotide sequence as shown in SEQ ID. NO 2.

6. The nucleic acid sequence according to claim 5, characterized in that it is coding for a polypeptide comprising 50% to 100%, preferably 100% sequence identity to a fragment or the entire sequence of the human Doll (hDoll-1) polypeptide as shown in SEQ. ID. NO 7.

7. The nucleic acid sequence according to claim 2, characterized in that it is the human doll-2 gene (hdoll-2) having the nucleotide sequence as shown in SEQ ID. NO 3.

8. The nucleic acid sequence according to claim 7, characterized in that it is coding for a polypeptide comprising 50% to 100%, preferably 100% sequence identity to a fragment or the entire sequence of the human Doll polypeptide (hDoll-2) as shown in SEQ. ID. NO 8.

9. The nucleic acid sequence according to claim 2, characterized in that it is the mouse doll-1 gene (mdoll-1) comprising the nucleotide sequence as shown in SEQ ID. NO 4.

10. The nucleic acid sequence according to claim 9, characterized in that it is coding for a polypeptide comprising 50% to 100%, preferably 100% sequence identity to a fragment or the entire sequence of the mouse Doll (mDoll-1) polypeptide as shown in SEQ. ID. NO 9.

11. The nucleic acid sequence according to claim 2, characterized in that it is the mouse doll-2 gene (mdoll-2) comprising the nucleotide sequence as shown in SEQ ID. NO 5.

12. The nucleic acid sequence according to claim 11, characterized in that it is coding for a polypeptide comprising 50% to 100%, preferably 100% sequence identity to a fragment or the entire sequence of the mouse Doll polypeptide (mDoll-2) as shown in SEQ. ID. NO 10.

13. A polypeptide being part of at least one signaling pathway in insects and vertebrates, characterized in that said polypeptide is the “Daughter of Legless” (DOLL) protein as well as homologues, fragments, derivatives and structural and functional analogs thereof.

14. The polypeptide according to claim 13, characterized in that said signaling pathway is the Wnt signaling pathway.

15. The polypeptide according to claim 14, characterized in that said polypeptide is the Drosophila melanogaster Doll protein having the amino acid sequence as shown in SEQ. ID. NO. 6.

16. The polypeptide according to claim 15, characterized in that said polypeptide has a 50% to 100%, preferably 100% sequence identity to a fragment or the entire sequence of the Doll polypeptide as shown in SEQ. ID. NO 6.

17. The polypeptide according to claim 14, characterized in that said polypeptide is the human Doll protein (hDOLL-1) having the amino acid sequence as shown in SEQ. ID. NO. 7.

18. The polypeptide according to claim 17, characterized in that said polypeptide has a 50% to 100%, preferably 100% sequence identity to a fragment or the entire sequence of the Doll polypeptide as shown in SEQ. ID. NO 7.

19. The polypeptide according to claim 14, characterized in that said polypeptide is the human Doll protein (hDoll-2) having the amino acid sequence as shown in SEQ. ID. NO. 8.

20. The polypeptide according to claim 19, characterized in that said polypeptide has a 50% to 100%, preferably 100% sequence identity to a fragment or the entire sequence of the Doll polypeptide as shown in SEQ. ID. NO 8.

21. The polypeptide according to claim 14, characterized in that said polypeptide is the mouse Doll-1 protein (mDoll-1) having the amino acid sequence as shown in SEQ. ID. NO. 9.

22. The polypeptide according to claim 21, characterized in that said polypeptide has a 50% to 100%, preferably 100% sequence identity to a fragment or the entire sequence of the Doll polypeptide as shown in SEQ. ID. NO 9.

23. The polypeptide according to claim 14, characterized in that said polypeptide is the mouse Doll-2 protein (mDoll-2) having the amino acid sequence as shown in SEQ. ID. 10.

24. The polypeptide according to claim 23, characterized in that said polypeptide has a 50% to 100%, preferably 100% sequence identity to a fragment or the entire sequence of the Doll polypeptide as shown in SEQ. ID. NO 10.

25. The nucleic acid sequence according to claim 2, characterized in that said nucleic acid sequence is coding for a polypeptide comprising a low overall amino acid sequence identity and a sequence identity of 50% to 100%, preferably 100% in conserved domains.

26. The nucleic acid sequence according to claim 25, characterized in that said conserved domain is the DHD domain.

27. The nucleic acid sequence according to claim 26, characterized in that said conserved domain is the PHD finger.

28. The nucleic acid sequence according to claim 2, characterized in that said sequence comprises a fragment of 20 to 100 nucleotides in length, preferably from 20 to 60 nucleotides, and most preferably from 20 to 50 nucleotides, said fragment being derived from SEQ. ID. NO. 1, 2, 3, 4 or 5.

29. The nucleic acid sequence according to claim 28, used as a hybridization probe.

30. A vector comprising a nucleic acid molecule encoding a Drosophila or vertebrate doll gene or a fragment thereof, selected from the group consisting of SEQ. ID. NO. 1, 2, 3, 4 or 5.

31. The vector according to claim 30, selected from the group consisting of eucaryotic and procaryotic expression vectors.

32. A host cell comprising the vector of claim 31, selected from the group consisting of mammalian cells, yeast cells, plant cells, insect cells or bacterial cells.

33. A method for the preparation of Doll proteins, fragments, derivatives and analogs thereof, comprising the steps of:
a) isolating a nucleic acid sequence containing the complete doll cDNA sequence or part thereof;
b) recombinantly expressing doll cDNA or a fragment thereof in bacterial, mammalian, plant, yeast or insect cells;
c) inducing protein production in said cells;
d) purifying Doll proteins.

34. The method according to claim 33, wherein step a) comprises the isolation of a nucleic acid molecule encompassing the doll cDNA from a vertebrate or invertebrate cDNA or genomic library.

35. The method according to claim 33, wherein step b) comprises the fusion of DNA encoding full-length or truncated doll to an epitope tag and a cleavage site contained within an inducible eucaryotic or procaryotic expression vector and transforming the appropriated host cells with said expression vector.

36. A chimeric protein comprising a Doll polypeptide fused to a heterologous amino acid sequence selected from the group consisting of an epitope-tagged sequence, an antibody, gluthathione-S-transferase protein, β-galactosidase, and alkaline phosphatase.

37. The chimeric protein according to claim 36, characterized in that the Doll polypeptide is selected from the group consisting of dDoll, hDoll-1, hDoll-2, mDoll-1 and mDoll-2.

38. The polypeptide according to claim 14, characterized in that it comprises the full length Doll polypeptide or a fragment thereof comprising an antibody-binding site for an anti-Doll antibody.

39. An antibody specifically recognizing a Doll polypeptide, said antibody being selected from the group consisting of polyclonal and monoclonal antibodies and fragments thereof.

40. An assay for studying diseases induced by a disrupted Wnt function or for drug screening comprising the use of organisms selected from the group consisting of Drosophila, mice, rats, rabbits, chicken, frogs, pigs or sheep, said organisms showing increased or reduced or no expression of doll or express a mutated Doll polypeptide in at least one tissue or organ.

41. The assay according to claim 40, characterized in that said organisms express the doll gene as a heterologous transgene.

41. The assay according to claim 40, characterized in that said doll gene comprises a mutation selected from the group consisting of deletions, point mutations, foreign DNA insertions and inversions.

42. Therapeutic and diagnostic method for the treatment of conditions selected from the group consisting of disorders of cell fate, differentiation or proliferation comprising the use of a Doll protein, homologue, derivative and fragment thereof.

43. Method of preparation of a therapeutic and diagnostic compound for the diagnosis of conditions selected from the group consisting of disorders of cell fate, differentiation or proliferation comprising the use of a Doll protein, homologue, derivative and fragment thereof.

44. Method of preparation of a therapeutic and diagnostic compound according to claim 43, characterized in that said therapeutic or diagnostic compound is selected from the group consisting of Drosophila and vertebrate Doll protein homologues and fragments thereof and antibodies and antibody fragments thereof.

45. Method of preparation of a therapeutic and/or diagnostic compound for the treatment of conditions selected from the group consisting of disorders of cell fate, differentiation or proliferation and its application to an individual comprising the use of doll nucleic acids, homologues, derivatives and fragments thereof.

46. Therapeutic and diagnostic method for the treatment of conditions selected from the group consisting of disorders of cell fate, differentiation or proliferation and its application to an individual comprising the use of doll nucleic acids, homologues, derivatives and fragments thereof.

47. Method of preparation of a therapeutic and/or diagnostic compound according to claim 45, characterized in that said therapeutic or diagnostic compound is selected from the group consisting of doll antisense DNA or RNA, doll double-stranded RNA and chemical or natural occurring compounds interfering with doll function.

48. A peptide comprising a fragment of the Doll polypeptide.

49. The peptide according to claim 48, comprising 40 to 60 amino acids in the N-terminal region of hDoll-1, hDoll-2, mDoll-1, mDoll-2 and Drosophila Doll.

50. The peptide according to claim 49, said peptide comprising the Doll homology domain (DHD).

51. Screening methods for the identification of chemical compounds, organic products, polypeptides or peptides interfering with Doll function in the Wnt pathway comprising the use of DHD.

52. Screening methods according to claim 51, wherein the screening assay based on protein-protein interactions.

53. Screening methods according to claim 52, wherein said screening methods are selected from the group comprising an in vitro protein-protein interaction assay and a protein-protein interaction assay in a host cell.

54. Screening methods according to claim 51, wherein the screening methods are for compounds specifically inhibiting the interaction between Doll and another protein.
